(19) United States
(12) Patent Application Publication
(10) Pub. No.: US 2023/0158300 A1
(43) Pub. Date: May 25, 2023

(54) PRECISION PHOSPHENE CONTROL THROUGH CUTANEOUS FACIAL ELECTRICAL STIMULATION

(71) Applicant: Ryerson University, Toronto (CA)

(72) Inventors: Alexandre DOUPLIK, North York (CA); Faraz SADRZADEH-AFSHARAZAR, Toronto (CA)

(21) Appl. No.: 17/996,909

(22) PCT Filed: Apr. 23, 2021
(86) PCT No.: PCT/CA2021/050557

§ 371 Date: Oct. 21, 2022

Related U.S. Application Data
(60) Provisional application No. 63/014,616, filed on Apr. 23, 2020.

(51) Int. Cl. A61N 1/36

(52) U.S. Cl. CPC A61N 1/36046; A61N 1/3603

(57) ABSTRACT

A device and method for passing information to a person using cutaneous facial stimulation to generate phosphenes is described. This invention has many uses, but in particular it can be used to transmit visuo-spatial information to a person who is blind or has impaired vision. Electrodes are attached to the face in known positions, and stimulated in an order to create a desired phosphene pattern in the user's field of vision. Masks are described to assist with holding the electrodes in place and to carry associated circuitry and sensors. Voltage waveform conditions are also identified to minimize pain associated with the use of the device.

### FIELD OF THE INVENTION

This invention relates to precise phosphene generation through non-invasive cutaneous electrical stimulation. Such localized phosphenes and phosphene patterns can be used to create a device to convey spatial or other information to a user through phosphenes. This would be useful, for example, to a person who has impaired vision.

### BACKGROUND OF THE INVENTION

Phosphenes refer to any visual images that are caused by any stimuli other than light. The most common stimulation of phosphenes are caused by mechanical means, such as when one rubs ones closed eyelids, which results in the perception of various star-like patterns. The most reproducible method for generating phosphenes is through electrical stimulation.

Gall et al., “Noninvasive transorbital alternating current stimulation improves subjective visual functioning and vision-related quality of life in optic neuropathy”, Brain Stimulation 175-188 (2011) proposes that the rate of phosphene perception is proportional to the frequency of the alternating current that is being applied. The stimulation was done by applying a square-wave current waveform bi-laterally across the eyeballs. Most people experience electrically induced phosphenes as spread-out yet localized flashes of light. The time of phosphene perception lines up with the rising and falling edges of the applied square wave. As the stimulation frequency goes up, the phosphenes start to fuse together due to the concept of persistence of vision—i.e. if a phosphene is induced by a current for 20 ms, the brain will perceive the phosphene for greater than 20 ms—and if multiple similar phosphenes are generated quickly and sequentially, the brain will start to perceive one persistent phosphene.

Sehic et al., “Electrical Stimulation as Means for Improving Vision”, American Journal of Psychology 186 (2016) suggests that applied current intensities of 300-900 μA are required to induce Phosphenes.

U.S. Pat. No. 9,254,385 by Greenberg et al. suggests the placement of an electrode array onto the retina to produce localized phosphenes. Even though this approach can produce excellent localized phosphenes, it involves surgical intervention and is highly invasive. Zalevsky et al., “Electromechanical tactile stimulation system for sensory vision substitution”, Optical Engineering 52 (2013) suggested the use of corneal electrode arrays for achieving highly resolved phosphenes. However, they did not account for the comfort and convenience of their intended users.

U.S. Pat. No. 2,703,344 by Anderson describes the use of cutaneous electrode arrays and various voltage waveforms to convey visual and auditory intelligence to the user. However, the invention utilizes vacuum tube technology, high voltages, and bulky magnetics. Similarly, U.S. Pat. No. 4,254,776 and 4,305,402 by Tanie et al. and Katims used cutaneous electrodes in combination with old technology (that cannot be made into a wearable device) and did induce phosphenes. Zeng et al., “Human Sensation of Transcranial Electric Stimulation” Scientific Reports 15247 (2019) used cutaneous electrodes to induce phosphenes and then attempted to spatially map their locations within the visual field. Their approach involved placing the electrodes on the scalp, which requires shaving patches of hair.

U.S. Pat. Nos. 4,664,117 and 4,979,508 by Beck propose the use of two electrodes to convey phosphenes to the user, for entertainment and aiding the visually impaired. His method for creating discriminable phosphenes was by moving the phosphenes within the visual field by manipulating waveform parameters such as amplitude, frequency, and duty-cycle. While he mentions the use of “four or more” electrodes, he only elaborates on the use of four or two electrodes. The microcontroller design from these patents uses dual in-line packaging that is not suited for implementation in wearables, due to its bulkiness.

Lastly, WO2013117800A1 by Vetek et al. suggests a digital approach to using phosphenes as a tool for conveying information to the user's visual field. This application uses analog means (i.e. transistors and op-amps) to generate the stimulation signals.

Pain and uncomfortable sensations may be caused by cutaneous electrical stimulation, which is undesirable. This concern can be relieved by at least six known mitigation strategies: (1) minimizing stimulation current (generally, the lower the better); (2) using two high frequency stimulation waveforms to form beats, as suggested by Oster et al. “Phosphenes,” Scientific American 83-87 (February 1970); (3) using pulse-width-modulation at a higher frequency, as the stimulation waveform, suggested by Gerasimenko et al. “Initiation and modulation of locomotor circuitry output with multi-site transcutaneous electrical stimulation of the spinal cord in non-injured humans,” Physiology-Heart and Circulatory Physiology 113 (February 2015); (4) strictly using bi-phasic pulses in order to leave no stray charges across the skin, suggested by Shinoda et al. “Transcutaneous electrical retinal stimulation therapy for age-related macular degeneration,” The open ophthalmology journal 2 (August 2008); (5) by minimizing the active pulse width to further reduce the applied dose; and (6) by using mild anesthetic agents at the sites of stimulation for creating a more comfortable experience.

### SUMMARY OF THE INVENTION

This application discloses that by running a voltage waveform through electrodes in pairs on specific locations on the skin of the face or body, phosphene patterns can be predictably produced in specific locations in a person's field of vision. Similarly, running voltage waveforms through electrodes in a monopolar configuration in specific facial locations will also produce phosphenes in predictable locations in the field of vision.

This insight can be used to create a device that uses phosphene patterns to communicate information (for example, spatial information about the wearer's surroundings) to the wearer. This device is non-invasive (i.e. it does not require surgery or connections directly to the optical nerve, brain or retina). This can be useful, for example, for a person who has a visual disability. General devices are described, but also specific embodiments are described that combine safety and practical effectiveness. It is also described how the pain associated with cutaneous stimulation of phosphenes can be reduced by limiting the pulse width, limiting the voltage waveform, and strictly controlling the waveform shape.

In accordance with the present invention, there is provided a system comprising: one to eight pairs of electrodes for cutaneous connection to a person, where the one to eight pairs of electrodes when connected to the person are located at a location selected from the group consisting of: the right temple of the person's head and the left temple of the person's head; or the left temple of the person's head and the left side of the person's head bilaterally across the nose bridge beneath the eyes; or the right temple of the person's head and the right side of the person's head bilaterally across the nose bridge beneath the eyes; or the right side of the person's head on the right cheekbone beneath the eyes to the right side of the nose bridge and the left side of the person's head on the left cheekbone beneath the eyes to the left side of the nose bridge; or the right side medially on the person's head and the left side medially on the person's head; or the left side medially on the person's head and the left side of the person's head on the left cheekbone beneath the eyes to the left side of the nose bridge; or the right side medially on the person's head and the right side of the person's head on the right cheekbone beneath the eyes to the right side of the nose bridge; or the right side laterally on the person's head and the left side distally on the person's head; each of one to eight pairs of electrodes being connected to a waveform generator; the waveform generators being connected to a first control device configured to activate a voltage waveform between the one to eight pairs of electrodes; and the system being connected to a power source.

In a first aspect of the invention, the one to eight pairs of electrodes are two to eight pairs of electrodes. In another aspect of the invention, the first control device being configured to receive input data and convert the data to a pattern of activation for the two to eight pairs of electrodes. In another aspect, the voltage waveform is a biphasic square signal of 1 to 30 Hertz and 10-500 μA. In another aspect of the invention, the voltage waveform is between 1 and 20 volts. In another aspect, the voltage waveform is between 10 and 15 Volts. In another aspect of the invention, the two to eight pairs of-electrodes and the waveform generators are part of a mask. In a further aspect, the first control device is part of the mask. In another aspect, the mask further comprises a sensor, the sensor being configured to send input data to the first control device. In another aspect, the sensor is a spatial sensor and the input data is spatial data and the first control device converts the spatial data into a phosphene pattern that, when the two to eight electrodes are connected to the person, communicates information about the person's spatial surroundings to the person.

In another aspect of the invention, the first control device is a digital computation device. In another aspect, the first control device comprises a graphics processor or a microcontroller or a single board computer. In still another aspect, the first control device limits the voltage waveform in each electrode pair bipolarly. In another aspect the first control device relays the signal across different electrode pairs through demultiplexing. In another aspect of the invention, each waveform generator comprises: a ripple rejector; a first half-bridge and a second half bridge together making a full bridge with a middle terminal; a bipolar current limiter connected in series with the first half-bridge, and the electrode attached to the waveform generator via the second half bridge. In another aspect of the present invention, the bipolar current limiter comprises two unipolar current limiters set in anti-parallel. In another aspect, the bipolar current limiter comprises a unipolar current limiter caged between the DC terminals of a full bridge rectifier. In another aspect of the invention, the system further comprises at least one additional pair of electrodes, the at least one additional pair of electrodes being connected to the waveform generator, and the first control device configured to activate a voltage waveform between the at least one additional pair of electrodes. In yet another aspect of this invention, the pattern of activation comprises activating at least two pairs of electrodes so as to produce centroids in the perception of a person who is connected cutaneously to the electrodes.

In accordance with the present invention, there is provided a system comprising: one to sixteen electrodes, where the one to sixteen electrodes when connected to a person are located at a location selected from the group consisting of: the left temple of the head; or the right temple of the head; or under the left eye at an interior position; or under the left eye at a middle position; or under the left eye at an exterior position; or under the right eye at an interior position; or under the right eye at a middle position; or under the right eye at an exterior position; or above the left eye at an interior position; or above the left eye at a middle position; or above the left eye at an exterior position; or above the right eye at an interior position; or above the right eye at a middle position; or above the right eye at an exterior position; or above the nose to the right side; or above the nose to the left side; where each of the one to sixteen electrodes form a unipolar pair with a seventeenth electrode, where the seventeenth electrode can be cutaneously connected to the person; each of the one to sixteen electrode pairs being connected to a waveform generator; the waveform generators being connected to a first control device configured to activate a voltage waveform between the each of the one to sixteen electrodes and the seventeenth electrode; and the system being connected to a power source.

In an aspect of this invention, the first control device is configured to receive input data and convert the data to a pattern of activation for the one to sixteen electrodes. In another aspect, the voltage waveform biphasic square signal of 1 to 30 Hertz and 10-500 μA. In another aspect, the voltage waveform is between 1 and 20 Volts. In another aspect, the voltage waveform is between 10 and 15 Volts. In yet another aspect, the one to sixteen electrodes and the seventeenth electrode and the waveform generators are part of a mask. In another aspect, the first control device is part of the mask. In still another aspect, the mask further comprises a sensor, the sensor being configured to send input data to the first control device. In still another aspect, sensor is a spatial sensor and the input data is spatial data and the first control device converts the spatial data into a phosphene pattern that, when the one to sixteen electrodes and the seventeenth electrode are connected to the person, communicates information about the person's spatial surroundings to the person. In another aspect, the first control device is a digital computation device. In another aspect of the present invention, the first control device comprises a graphics processor or a microcontroller or a single board computer. In another aspect of the invention, the pattern of activation comprises activating at least two pairs of electrodes so as to produce centroids in the perception of a person who is connected cutaneously to the electrodes.

In accordance with the present invention, there is provided a method of generating phosphenes in a person, comprising: cutaneously attaching one to eight pairs of electrodes to a person, where the one to eight pairs of electrodes whose locations are selected from the group consisting of: the right temple of the person's head and the left temple of the person's head; or the left temple of the person's head and the left side of the person's head bilaterally across the nose bridge beneath the eyes; or the right temple of the person's head and the right side of the person's head bilaterally across the nose bridge beneath the eyes; or the right side of the person's head on the right cheekbone beneath the eyes to the right side of the nose bridge and the left side of the person's head on the left cheekbone beneath the eyes to the left side of the nose bridge; or the right side medially on the person's head and the left side medially on the person's head; or the left side medially on the person's head and the left side of the person's head on the left cheekbone beneath the eyes to the left side of the nose bridge; or the right side medially on the person's head and the right side of the person's head on the right cheekbone beneath the eyes to the right side of the nose bridge; or the right side laterally on the person's head and the left side distally on the person's head; where each of the one to eight pairs of electrodes is connected to a waveform generator; and the waveform generators are connected to a first control device configured to activate a voltage waveform between the one to eight pairs of electrodes; and sending input data to the first control device, the first control device converting the input data to a pattern of activation for the one to eight pairs of electrodes, and so activating the one to eight pairs of electrodes to generate a phosphene pattern in the person.

In a first aspect of this invention, the one to eight electrode pairs are two to eight electrode pairs. In another aspect of this invention, the voltage waveform is a biphasic square signal of 1 to 30 Hertz and 10-500 μA. In another aspect, the voltage waveform is between 1 and 20 Volts. In another aspect if the invention, the voltage waveform is between 10 and 15 Volts. In another aspect, the two to eight pairs of electrodes and the waveform generators are part of a mask and the mask is worn by the person. In another aspect the first control device is part of the mask. In still another aspect, the mask further comprises a sensor, the sensor being configured to send input data to the first control device. In another aspect, the sensor is a spatial sensor and the input data is spatial data and the phosphene pattern communicates information about the person's spatial surroundings to the person. In another aspect, the first control device is a digital computation device. In another aspect, the first control device comprises a graphics processor or a microcontroller or a single board computer. In still another aspect, the first control device limits the voltage waveform in each electrode pair bipolarly. In still another aspect, the first control device relays the signal across different electrode pairs through demultiplexing. In another aspect, each waveform generator comprises: a ripple rejector; a first half-bridge and a second half bridge together making a full bridge with a middle terminal; a bipolar current limiter connected in series with the first half-bridge, and the electrode attached to the waveform generator via the middle terminal. In another aspect the bipolar current limiter comprises two unipolar current limiters set in anti-parallel. In another aspect, the bipolar current limiter comprises a unipolar current limiter caged between the DC terminals of a full bridge rectifier. In another aspect of the invention, the pattern of activation comprises activating the one to eight pairs of electrodes so as to produce centroids in the perception of a person who is connected cutaneously to the electrodes.

In accordance with the present invention, there is provided a method of generating phosphenes in a person, comprising: cutaneously connecting one to sixteen electrodes to a person, where the one to sixteen electrodes whose locations are selected from the group consisting of: the left temple of the head; or the right temple of the head; or under the left eye at an interior position; or under the left eye at a middle position; or under the left eye at an exterior position; or under the right eye at an interior position; or under the right eye at a middle position; or under the right eye at an exterior position; or above the left eye at an interior position; or above the left eye at a middle position; or above the left eye at an exterior position; or above the right eye at an interior position; or above the right eye at a middle position; or above the right eye at an exterior position; or above the nose to the right side; or above the nose to the left side; where each of the one to sixteen electrodes form a unipolar pair with a seventeenth electrode, where the seventeenth electrode is cutaneously connected to the person; and each of the one to sixteen electrodes is connected to a waveform generator; the waveform generators being connected to a first control device configured to activate a voltage waveform between the each of the one to sixteen electrodes and the seventeenth electrode; and sending input data to the first control device, the first control device converting the input data to a pattern of activation for the one to sixteen electrodes, and so activating one to sixteen electrodes to generate a phosphene pattern in the person.

In another aspect of the present invention, the voltage waveform is a biphasic square signal of 1 to 30 Hertz and 10-500 μA. In another aspect, the voltage waveform is between 1 and 20 Volts. In another aspect, the voltage waveform is between 10 and 15 Volts. In another aspect, the one to sixteen electrodes and the waveform generators are part of a mask and the mask is worn by the person. In yet another aspect, the first control device is part of the mask. In still another aspect, the mask further comprises a sensor, the sensor being configured to send input data to the first control device. In another aspect, the sensor is a spatial sensor and the input data is spatial data and the phosphene pattern communicates information about the person's spatial surroundings to the person. In another aspect of the invention, the pattern of activation comprises activating the one to sixteen electrodes so as to produce centroids in the perception of a person who is connected cutaneously to the electrodes.

In accordance with the present invention, there is provided a method of generating centroids in a person, comprising activating two or more pairs of electrodes cutaneously attached to the person, and varying the intensity of the activated two or more pairs of electrodes to locate the centroid in the person's visual field.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates the location of electrodes on the face in a first embodiment, as well as the wiring diagram of eight differential waveform generators.

FIGS. 2a and 2b illustrate electrode multiplexing permutations and the locations of the phosphenes they cause in the visual field when applied.

FIG. 3 is a front view of a mask to control the location of and hold the electrodes to the face of a user.

FIG. 4 is a high-level illustration of the processing flow for the phosphene stimulation device.

FIG. 5 is a condensed/modularized circuit diagram of a preferred embodiment of the phosphene stimulation device.

FIG. 6 is a generalized illustration of the circuitry used in this invention.

FIG. 7a is an illustration of electrode placement in a second embodiment of the phosphene stimulation device; and

FIG. 7b is a simplified lumped wiring diagram of the second embodiment of the phosphene stimulation device showing sixteen differential waveform generators.

FIG. 8 is an illustration of the location of the phosphenes created in the visual field when unipolar electrodes as located in FIG. 7a are activated.

FIG. 9 is an illustration of different binocular perimetry targets.

FIG. 10 is an illustration of observed correlations between activation of electrodes in certain locations and the phosphenes generated as illustrated on perimetry targets.

FIG. 11 is an illustration of the experimental setup used in a pilot study.

FIG. 12 is an illustration of electrode placement, stimulation current and channels used in experiments.

FIG. 13 is an illustration of the experimental setup.

FIG. 14 is an illustration of the circuit architecture of the stimulus administrator.

FIG. 15 is an illustration of the pre-processing and post-processing process flow to get the raw phosphene drawings to individual and population phosphene maps, and then to the Receive Operating Characteristic (ROC) and centroid analysis stage.

FIG. 16 is an illustration of an example of the ROC analysis being computed on one of the population phosphene maps.

FIG. 17 is a chart showing the efficiency of each stimulation channel as well as the control in stimulating phosphenes.

FIG. 18 is an illustration of mass centroid discriminability, comparing left vs. right to up vs. down.

FIG. 19 is an illustration of perimetry phosphene density for all population phosphene maps.

FIG. 20a is a table with results from the statistical analysis of the experimental results.

FIG. 20b is the population phosphene maps for CH1 through CH7.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. Those of skill in the art understand that other embodiments may be utilized and structural changes may be made without departing from the scope of the present invention. Likewise, a reasonably broad scope for claimed or covered subject matter is intended. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

This invention stimulates phosphenes within the visual field of a person through the use of electrical stimulation on the skin, as means of providing artificial vision. The electrical stimulation takes place on the face, using two or more cutaneous surface electrodes. In a preferred embodiment, described below, eight cutaneous surface electrodes are used, where the electrodes are activated in bi-polar pairs. In another embodiment described below, 16 cutaneous surface electrodes are used, which are each tied to a single opposite pole and can each by activated individually as mono-poles. In all cases, a mask or visor can be used to place and hold the electrodes to the skin.

To minimize pain associated with the activation of the electrodes, a biphasic square signal of 1 to 30 Hertz is used. As noted below, it is preferred that the voltage waveform be between 0 and 20 Volts, and in a more preferred embodiment between 10 and 15 Volts. The Voltage Waveform should be between 10-500 μA. Experiments have shown that to minimize pain, it is important that the signal be biphasic and a strict square signal. To the inventor's knowledge, previous investigations have used sinusoidal-wave, triangular wave or monophasic signals resulting in higher levels of user pain.

Activating specific electrode pairs (whether bi-polar pairs or mono-polar pairs) will generate phosphenes in predictable positions in the user's field of vision. By selecting which pairs to activate, information can be passed to the user through the generation of phosphenes.

Generally, any information can be passed to the user through phosphenes—for example, a user working in a dangerous environment could receive information about danger or safety levels as determined by a third party observer. In one potential application, the information is spatial information about a user's immediate surroundings, which is of particular usefulness if the user is blind or has a vision impairment.

Generally, whatever information that needs to be passed to the user needs to be translated into a pattern of electrode pair activations (which can also be called pattern generation). This translation or pattern generation can be accomplished through various types of circuitry and/or software. In a preferred embodiment, this pattern generation is accomplished by using a graphics processor with multiple parallel processors, allowing translation/pattern creation in real time. The user will also have to understand the meaning of the generated phosphenes: in some cases the meaning may be intuitive (such as indicating spatial barriers), but in other cases specific meanings may be associated with specific phosphenes.

In a first embodiment, this invention uses facial cutaneous electrode pair locations that, when a voltage waveform is applied across the locations (i.e. they are a bipolar pair), creates a phosphene in a reproducible location in a person's field of vision. The response time between the application of the voltage waveform and the appearance of the phosphene is on the scale of 200 milliseconds. While in principle any number of two or more electrodes may be used to create a plurality of potential electrode pairs, it is considered that eight electrodes provides a desirable range of phosphene generation. By locating eight electrodes on the face in specific different locations, the loci of the stimulation can be changed by multiplexing a voltage waveform across different electrodes to create a desired phosphene pattern.

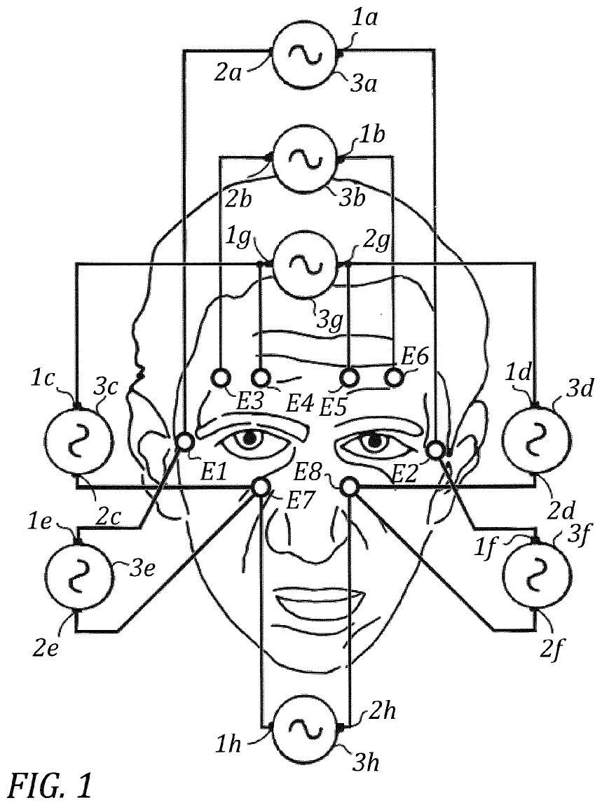

The eight electrodes are wired to eight digital signal generators, as depicted in FIG. 1. In a preferred embodiment, the signal generators are galvanically isolated. In another preferred embodiment, the signal generators are galvanically isolated four-quadrant current-limited digital signal generators. The signal generators are set up differentially, where each can output a positive, negative, or bi-phasic pulse. The signal generators can also be set into an off state, where no potential appears across the differential pair. The signal generators are controlled in unison, using a parallel interface with the graphics processor. In a first embodiment, the multiplexing between electrodes entails keeping one differential pair active at times, while keeping the rest in the off state. In another embodiment, the multiplexing allows for more than one differential pair to be active at the same time, producing more than one phosphene. A controller strategically alternates the active state across the eight differential pairs to create a phosphene pattern. Since the differential pairs are wired to various designated locations on the face, the multiplexing of pulses across different electrodes, changes the stimulation loci by re-routing the voltage waveform pathways in the head.

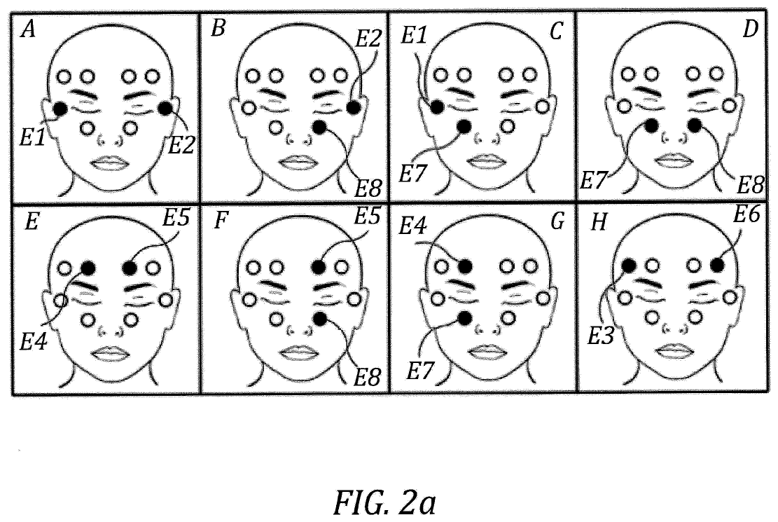

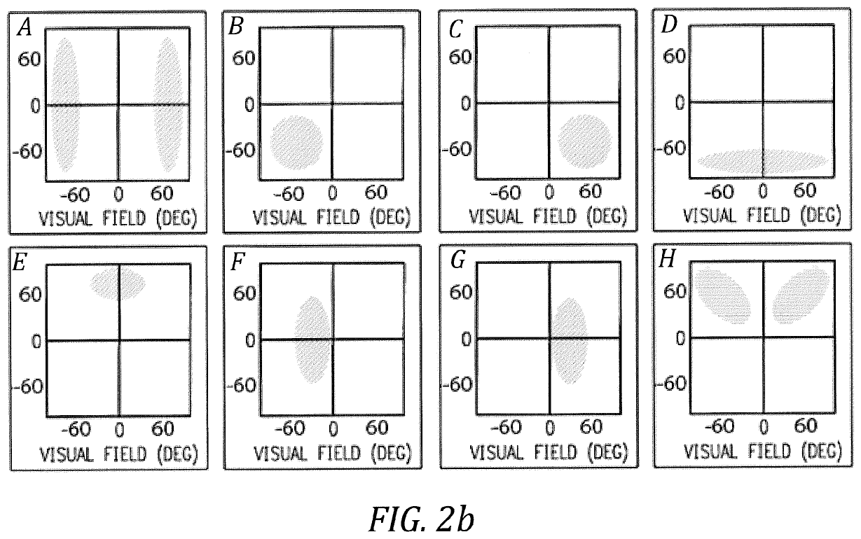

The phosphenes that result from cutaneous electrical stimulation have perceptive properties such as: shape, size, intensity (brightness), location within the visual field, and flicker frequency. The mapping between the electrode location and phosphene properties is consistent among the tested population. The observed electrode location versus phosphene visual field location is outlined in FIG. 2.

A multiplicity of such phosphenes can be induced in a quick bursting action, to draw primitive images onto the visual field, due to persistence of vision. This can be referred to as a compound phosphene. The user can be trained to: 1) identify and discriminate various phosphenes and compound phosphenes 2) associate each with information, such as a warning of danger or an implicit notion of navigation/spatial perception, and 3) if necessary, to act upon such information.

The controller (which in a preferred embodiment is a graphics processor) should be able to processes the sensor data or other input data and output stimulation pulses to create phosphene patterns in real-time. In a preferred embodiment, a graphics processor is used that possesses many processing cores, allowing for parallel processing, resulting in very-low latency processing.

As noted above, to minimize pain the signals or voltage waveform, between the electrodes should be a biphasic square signal of 1 to 30 Hertz. It is preferred that the voltage waveform be between 0 and 20 Volts. In a more preferred embodiment, the voltage waveform is between 10 and 15 Volts. The voltage waveform intensities should be kept to a minimum, between 10-500 μA. It is assumed that the threshold voltage waveform in μA to generate phosphenes can vary in a population. A standardized method can be used to identify this threshold for each individual: initially, a stimulation waveform is applied at 10 μA peak, and the intensity is slowly raised until the user perceives vivid phosphenes. However, the voltage waveform intensity should always kept below 500 μA. It is not recommended that Individuals who fail to see phosphenes at 500 μA use the invention as described herein, since higher voltage waveform intensities might be dangerous in the long run. The setups of the first and second embodiments minimize the voltage waveform path length in the body, thereby reducing the applied EM dose. However, proper low-frequency EM dose calculations should be tracked to keep the voltage waveform dose within safe limits. This is particularly important when phosphene stimulation is to be applied for long time spans.

FIG. 1 illustrates the location of electrodes on the face in a first embodiment of the invention, as well as the wiring diagram of eight differential waveform generators. Turning to FIG. 1, eight skin electrodes E1, E2, E3, E4, E5, E6, E7, and E8 are positioned onto the head on the illustrated locations (E1 . . . E8). Skin electrode E6 is positioned on the left side distally on the forehead and electrode E3 on the right side laterally on the forehead, and skin electrode E4 is positioned on the right side medially on the forehead and E5 is positioned on the left side medially onto the forehead. Skin electrode E7 is positioned on the right side of the person's head on the right cheekbone beneath the eyes to the right side of the nose bridge, and electrode E8 is positioned on the left side of the person's head on the left cheekbone beneath the eyes to the left side of the nose bridge. Skin electrodes E1 and E2 are positioned onto the right and left temples of the head respectively.

These skin electrodes are connected to waveform generators 3a, 3b, 3c, 3d, 3e, 3f, 3g and 3h that are wired their respective skin electrodes. Each waveform generator is associated with cathode 1a, 1b, 1c, 1d, 1e, 1f, 1g and 1h and anode 2a, 2b, 2c, 2d, 2e, 2f, 2g or 2h. Specifically, waveform generator 3a is wired differentially across E1 and E2; waveform generator 3b is wired differentially (i.e. no common grounding point) across E3 and E6; waveform generator 3c is wired differentially across E4 and E7; waveform generator 3d is wired differentially across E5 and E8; waveform generator 3e is wired differentially across E1 and E7; waveform generator 3f is wired differentially across E2 and E8; waveform generator 3g is wired differentially across E4 and E5; and waveform generator 3h is wired differentially across E7 and E8. Electrodes E1 through E8 can be any form of cutaneous electrode.

In one embodiment, the waveform generators 3a through 3h are current-limited four-quadrant digital waveform generators.

As known to person skilled in the art, the skin electrodes may be adhered to the skin using any suitable electrode gel such as Ten20™ EEG electrode gel, in order to lower the skin impedance.

By activating a voltage waveform between pairs of the electrodes E1 through E8, phosphenes may be reliably generated in the visual field of the person connected to the electrodes. FIGS. 2a and 2b illustrate electrode multiplexing permutations and the phosphenes they cause in the visual field when applied. Turning to FIG. 2a, boxes A through H show examples of activation of pairs of the electrodes E1 . . . E8 from FIG. 1, where black filled circles represent active electrodes and hollow circles represent inactive electrodes. Each pair of activated electrodes will create a phosphene in a predictable location as illustrated in FIG. 2b. Turning to FIG. 2b and referring to FIG. 2a, as seen in box A in FIG. 2a and box A in FIG. 2b, when electrodes E1 and E2 are activated, phosphenes are generated in the wearer's visual field as seen in box A in FIG. 2b.

As seen in box B in FIG. 2a and box B in FIG. 2b, when electrodes E2 and E8 are activated, phosphenes and generated in the wearer's visual field as seen in box AB in FIG. 2b.

As seen in box C in FIG. 2a and box C in FIG. 2b, when electrodes E1 and E7 are activated, phosphenes and generated in the wearer's visual field as seen in box C in FIG. 2b.

As seen in box D in FIG. 2a and box D in FIG. 2b, when electrodes E7 and E8 are activated, phosphenes and generated in the wearer's visual field as seen in box D in FIG. 2b.

As seen in box E in FIG. 2a and box E in FIG. 2b, when electrodes E4 and E5 are activated, phosphenes and generated in the wearer's visual field as seen in box E in FIG. 2b.

As seen in box F in FIG. 2a and box F in FIG. 2b, when electrodes E5 and E8 are activated, phosphenes and generated in the wearer's visual field as seen in box F in FIG. 2b.

As seen in box G in FIG. 2a and box G in FIG. 2b, when electrodes E4 and E7 are activated, phosphenes and generated in the wearer's visual field as seen in box G in FIG. 2b.

As seen in box H in FIG. 2a and box H in FIG. 2b, when electrodes E3 and E6 are activated, phosphenes and generated in the wearer's visual field as seen in box H in FIG. 2b.

In FIG. 2b, the numbers 60 and −60 represent degrees in the field of vision. The majority of the visual perception for people lies within these degrees (taking into account peripheral vision).

FIGS. 2a and 2b represent known electrode locations and associated phosphene locations. It is believed that additional pairs of electrode locations may be determined that will consistently generate phosphenes in a predictable manner—these may be added to the inventive device and methods in future embodiments of the invention.

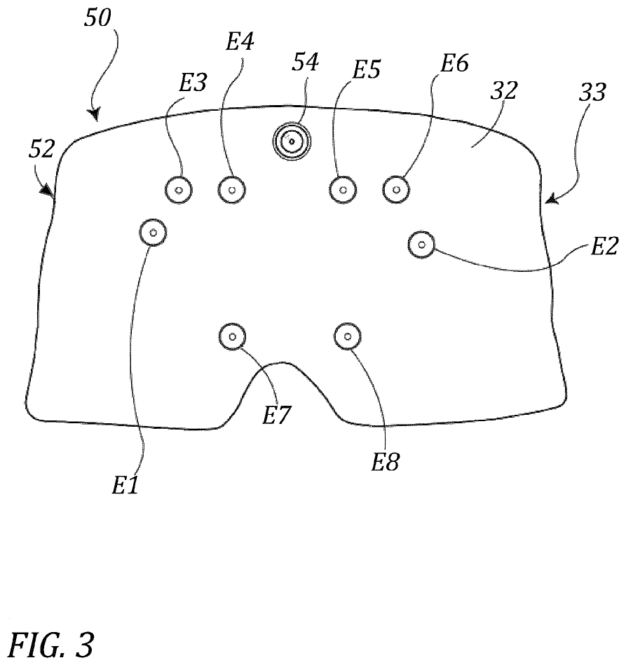

FIG. 3 is a front view of a mask to reproducibly control the location of and hold the electrodes to the face of a wearer. Turning to FIG. 3, there is a mask 50, which is held onto the user's face by back straps 7. In a preferred embodiment, the mask will be made from double-layered soft fabric, possibly elastic, where the electrodes E1, E2, E3, E4, E5, E6, E7 and E8 are integrated into the mask. The electrodes will be connected to the fabric mask, in any suitable manner known to persons of skill in the art, such as using grommets. In one embodiment, the electrodes E1, E2, E3, E4, E5, E6, E7 and E8 are each cup shaped and have a hole at the center, where electrode gel can be injected. In another embodiment, the electrodes are selected to not require the use of electrode gel.

A sensor 54 may optionally be located somewhere on the mask 50. In one embodiment, the sensor can sense spatial surroundings. In one embodiment, the sensor 54 is a camera, and in a preferred embodiment is similar to the cameras used in smartphones. In the embodiment of FIG. 3, the sensor 54 is located at the center of the upper edge of the mask. In another embodiment, more than one sensor is located on the mask and sends inputs into the phosphene pattern generation system.

As illustrated, mask 50 does not have any holes for eyes or ears. Such eyeholes or earholes may optionally be included in mask 50.

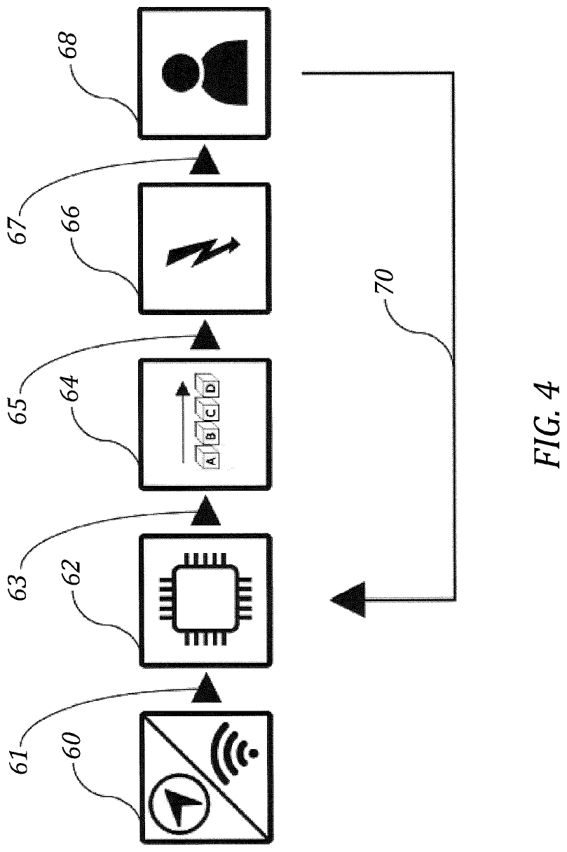

FIG. 4 is a high-level illustration of the processing flow for the phosphene stimulation device. Turning to FIG. 4, there is a process 60 to create input data 61. Process 60 can include any process that generates input data 61 to be input into the system and translated to a phosphene pattern. In one embodiment, process 60 is a person inputting information to be sent to the user of the system. In another embodiment, process 60 involves one or more sensors that send information to the system via input data 61. The sensors generally do not have to be part of a mask or carried by the user of the inventive system. In a specific embodiment, the sensor senses the spatial environment of the wearer: in a simple example, step 60 is implemented using a camera. There can also be a multiplicity of sensors used to generate the input data. In step 62, the input data 61 is processed to create phosphene data 63 that reflects what phosphenes should be generated—i.e. a phosphene pattern. In a particular embodiment, the phosphene patterns are designed to reflect the wearer's spatial environment. In step 64, the phosphene data 63 is transformed into a sequence 65 of multiplexing instructions for electrode activations—the activation of paired electrodes with specific waveforms—to produce the desired phosphene pattern. In step 66, the activation of paired electrodes with specific waveforms is performed, resulting in the user in step 68 experiencing the phosphene pattern. Finally, in step 70 the user provides feedback on the performance of the system which can be used to adjust steps 60, 62, 64 and 66.

Ideally, feedback step 70 is used in a training period to adjust the device, but once this training is performed the device will need minimal adjustment.

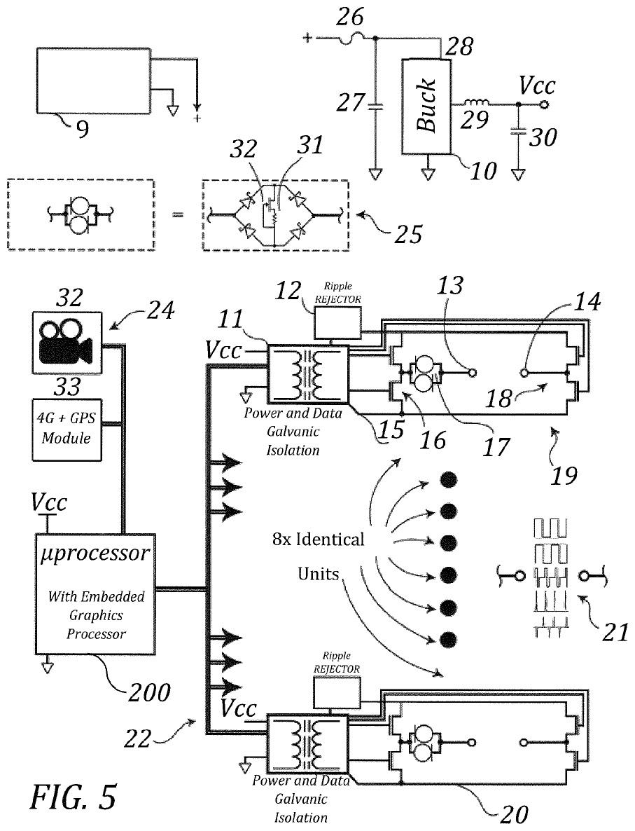

FIG. 5 is a detailed modularized circuit diagram of a preferred embodiment of the phosphene stimulation device. Turning to FIG. 5, the device is powered from a 9V battery 9. In a preferred embodiment, a lithium battery can be used, in conjunction with battery charging/protection circuitry. The battery input is passed through a fuse 26, buffered by an input capacitor 27, then stepped down by a buck converter 28 to 5 Volts. The buck converter's output ripple is smoothened by the output LC filter 29, 30. This generates a 5V regulated output Vcc. The Vcc feeds the main graphics processor 200, camera 32, and the 4G/GPS module 33. The 4G/GPS module 33 features an antenna that extends its range of communication. The camera 32 and the 4G/GPS module 33 stream is fed into the graphics processor 200. The graphics processor 200 outputs to the eight waveform generators in a multiplexed fashion (i.e. a queue of sequential activation of electrode pairs).

A waveform generator is included in FIG. 5. The signal passes through galvanic isolation unit 11, which isolates power and data from the other waveform generators. Thus, no voltage waveform can flow between the isolated channels. In FIG. 5, embodiments 15 and 20 represents two galvanically isolated ground potentials, for two different waveform generators for two different stimulation channels or electrode pairs. (This will also eliminate a path to the ground, thus reducing chances of electrocution if the system is connected to a power source other than an everyday battery). As discussed above, it is important that the waveform generator include a ripple rejector, since the use of a strict step waveform will reduce pain to the user. In this embodiment, bypassing by a LC filter followed by a transistor-based capacitor multiplier circuit, the ripple noise is eliminated, and a clean DC source is created (ripple rejector 12). At this point, eight galvanically isolated ripple-free 15V rails are created. In order to convert the DC rail, into a four-quadrant signal source, half bridges 16 and 18 are used, which together form a full bridge with a middle terminal. In order to implement current limiting circuitry, a bi-directional current limiter 17 is placed in series with one of the half-bridge terminals 16. The current limiter consists of a full-bridge rectifier where the AC terminals are connected in series with the half-bridge terminal 16 and the DC terminals are connected to a unidirectional current limiter 25. The unidirectional current limiter is a well-known circuit that uses a depletion-mode N-channel JFET or MOSFET 32, with a resistor 31 connected between the gate and the source. The voltage drop across the resistor, places a negative bias voltage on the gate, which drives the FET into saturation mode, thus limiting the current across the drain and the source. The resistor 31 can be modified to set the current limit. In operation, one of the electrodes off the activated electrode pair is connected to node 13 which connects to the current limiter circuitry, while node 14 (via the middle terminal of the full bridge) is connected to the other electrode in the pair. Through this setup, various waveforms can be outputted from each stimulation channel/electrode pair, which include: positive square-wave, negative square-wave, bi-phasic square-wave, mono-phasic pulse, and bi-phasic pulse. Arbitrary bi-polar waveforms can be created, through the use of beat formation and pulse-width-modulation.

This wave-form generator can be used to create a sharp square signal, and can create a bi-phasic signal in accord with this invention. Persons skilled in the art will understand that other forms of circuitry and/or software can be used to similar effect.

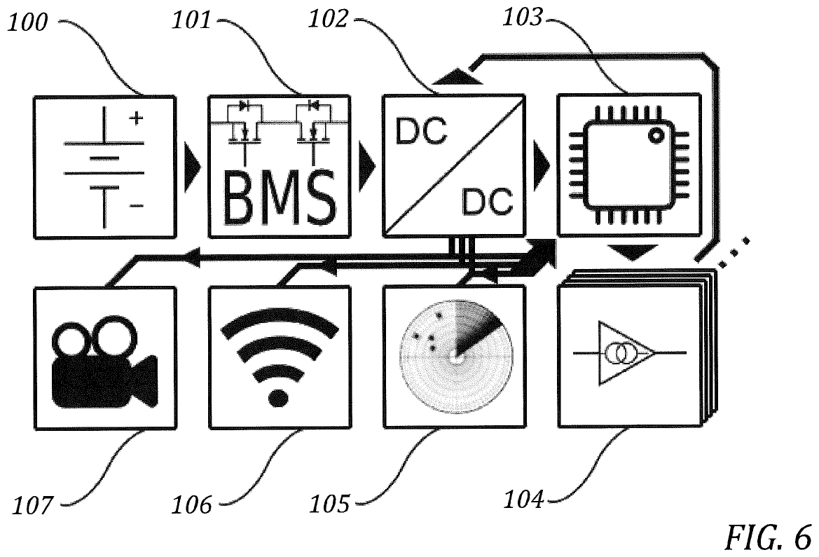

FIG. 6 describes a generalized form of circuitry for a particular wearable embodiment of the invention. As depicted in FIG. 6, this embodiment includes some form of power source 100. The most conventional power source for wearables is a battery. This embodiment includes a Battery Management System (BMS) 101. The BMS block serves to ensure the long-term functionality of the battery. This unit protects the battery from being charged or discharged too much. The unit can also safely charge the battery, when connected to a power source. The unit 102 converts the potential difference generated by the battery into a potential difference appropriate for the operation of a central processing unit 103, as well as for the operation of a stimulation administrator 104. The central processing unit 103 simultaneously communicates with sub-modules, responsible for spatial sensing 105, network connectivity 106, and video capturing 107. The central processing unit 103 has multiple memory buffers and can accommodate the data bandwidth from the mentioned sensors. Using this info, the central processing unit sends signals to the stimulation administrator 104, telling it the waveform shape, intensity, and channel selection. The stimulation administrator 104, following the command of the central processing unit 103, generates a precise current limited stimulation voltage waveform in the chosen channel or electrode pair and so stimulates the user to create phosphenes. In alternatives to this embodiment, submodules 105, 106, and 107 may not be present, although there must be some sort of input into the central processing unit 103 to be converted into a phosphene pattern.

A second approach to phosphene stimulation uses mono-polar electrodes as compared to the previous embodiments which used bi-polar or paired electrodes. In this approach, all of the stimulation signals (or, equivalently, each electrode) shares a common ground node. The ground node can be placed onto any part of the body.

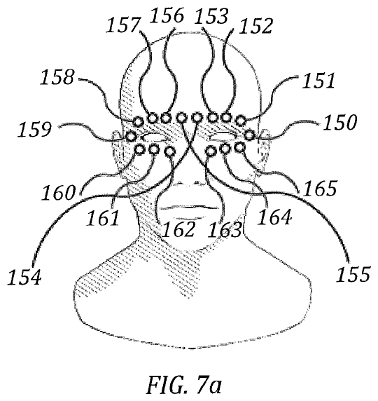

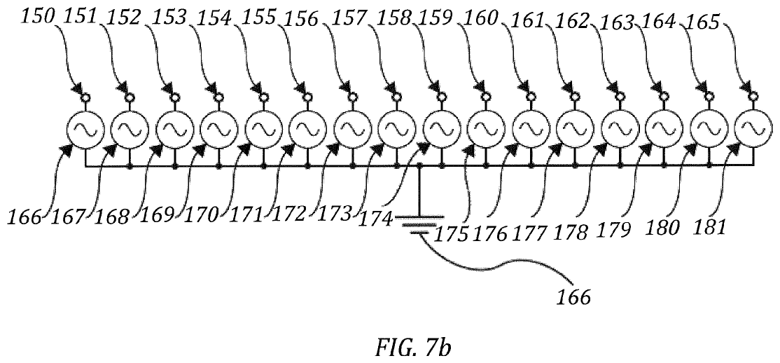

One embodiment of a mono-polar system is illustrated in FIGS. 7a and 7b. Turning to FIG. 7a, the placement of electrodes 150 to 165 are illustrated. In a particular embodiment, a mask is used that when worn by the user touches electrodes to the skin in locations 150-165. The placement of grounding electrode 166 is not illustrated since it could be placed anywhere. In a preferred embodiment, the grounding electrode 166 is placed in the back of the ears. Signal generators 166 to 181 are connected to electrodes at locations 150-165 as illustrated in FIG. 7b. The signal generators 166-181 can produce any positive or negative voltage and have current limiting capabilities. In a preferred embodiment, the signal generators 166-181 generate a 10-15 Volt biphasic square signal of 1 to 30 Hertz, where the voltage waveform is between 10-500 μA.

More specifically, location/electrode 150 is located on the left temple of the head, and location/electrode 159 is located on the right temple of the head. Location/electrodes 163, 164 and 165 are located under the left eye at an interior position 163, middle position 164 and exterior position 165. Location/electrodes 162, 161 and 160 are located under the right eye at an interior position 162, middle position 161 and exterior position 160. Location/electrode 155 is located above the nose to the right side, and location/electrode 154 is located above the nose to the right side. Location/electrodes 156, 157 and 158 are located above the right eye at interior position 156, middle position 157 and exterior position 158. Location/electrodes 153, 152 and 151 are located above the left eye at interior position 153, middle position 152 and exterior position 151.

Each of the electrodes 150 to 165 will, when activated, produce a phosphene in a specific location in the field of vision. Two phosphenes will appear if two electrodes are activated simultaneously, although this may be perceived by the wearer as one phosphene.

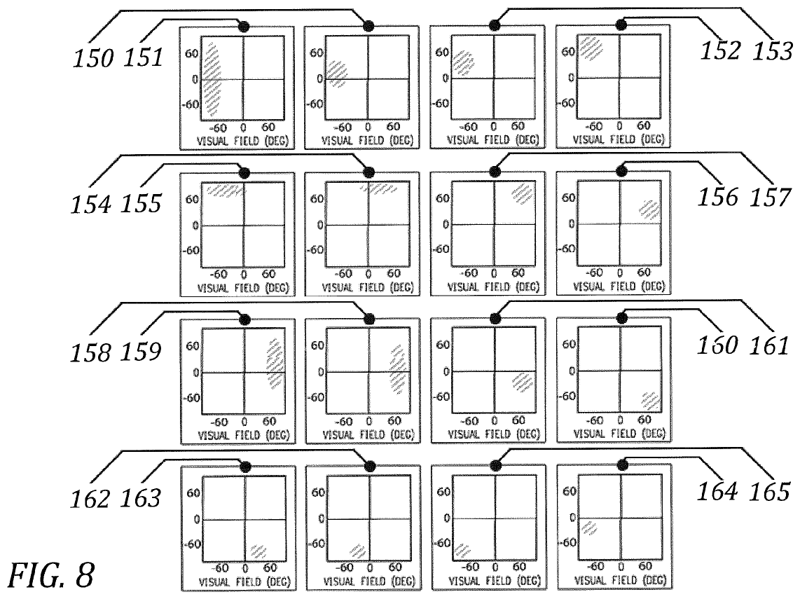

FIG. 8 illustrates the location of the phosphenes resulting from the stimulation of mono-polar electrodes 150-165. It is expected that this correspondence will hold true for the majority of the population; however, in some cases there may be variations.

The mono-polar electrode embodiment described above may be implemented using a mask to control the location of the electrodes on the face, similar to the mask described above. One advantage of the mono-polar electrode approach is that the corresponding wave-form generators will be less bulky than in the bi-polar case, largely because there is no need to galvanically isolate the electrodes. As a result, a mono-polar mask that includes the associated circuitry can be much less bulky than a mask for use with a bi-polar system, or equivalently many more mono-polar electrodes can be placed within a similarly bulky mask.

### Integrated Phosphene Stimulated Perception

Whether using mono-polar electrodes or bi-bolar (pairs of) electrodes, the activation of more than one pair, either simultaneously or close in time, and with varying current intensities, can be used to create and control the location of phosphenes across the entire visual field, which may be called integrated phosphene stimulated perception.

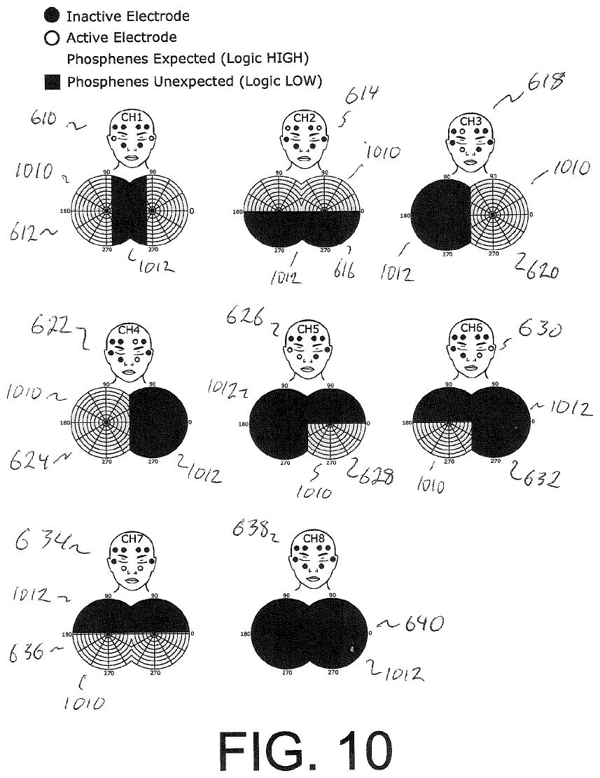

This can be achieved through the stimulation of two or more pairs of electrodes, as illustrated in FIG. 10, which leads to the perception of phosphenes (which we will call centroids) at a central location between the phosphenes usually generated by the electrode pairs when activated one at a time. For instance, if the stimulation of a first pair of electrodes A alone produces a phosphene at location X and the stimulation of a second pair of electrodes B alone produces a phosphene at location Y, the simultaneous stimulation of electrode pairs A and B produces a phosphene (or centroid) centered about Z, with Z being the geometric midpoint of locations X and Y.

The above example assumes that the stimulation by electrode pairs A and B have identical electrical stimulation intensity. (Intensity means power per area) Increasing the intensity applied by one electrode pair relative to the other electrode pair produces a spatially offset centroid that is proportionally closer to the location of the phosphene generated by the higher intensity electrode pair when the higher intensity electrode pair is activated alone. To exemplify the second approach, assume that the stimulation of a first pair of electrodes A alone produces a phosphene at location X, and the stimulation of a second pair of electrodes B alone produces a phosphene at location Y. Simultaneously activating electrode pairs A and B, while having electrode pair A administered at twice the intensity of electrode pair B, results in a centroid perception that is proportionately closer to X than Y.

By using the approaches in the preceding paragraphs, centroids/phosphenes can be reliably and predictably generated across the entire visual field. As a result, more complex and detailed imaging through phosphenes can be perceived across the entire visual field using the convenient apparatus already described.

### Experimental Results

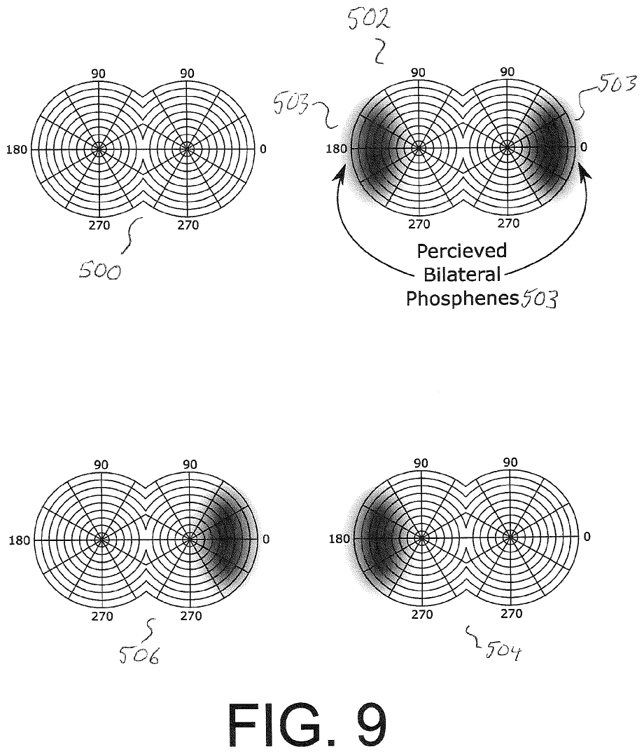

Phosphenes are an experienced phenomenon and are hard to measure or even register externally. Therefore, subjects must introspectively experience and perceive phosphenes and then describe their experience through an oral description or drawings. FIGS. 9 and 10 summarize the electrode configuration, experimental setup, and the truth maps of the expected hypothesized phosphenes, for the conducted human study.

The perception of phosphenes is represented on a binocular perimetry target, which we will refer to as a perimetry target. Turning to FIG. 9, a perimetry target where there is no stimulation is labelled 500. A perimetry target where there is alternating current stimulation over the facial surface across the temples is labelled 502. Note that in perimetry target 502, there are perceived bilateral phosphenes 503. Perimetry target 504 shows a left phosphene, which can be interpreted as the left direction. Perimetry target 506 shows a right phosphene, which can be interpreted as the right direction.

Turning to FIG. 10, there is an illustration of observed correlations between activation of electrodes in certain locations and the phosphenes generated as illustrated on perimetry targets. Turning to FIG. 10, for the active electrode placement as illustrated in illustration 610, there is a perimetry target of 612, which we propose to indicate forward. For the active electrode placement as illustrated in illustration 614, there is a perimetry target of 616 which we propose to indicate ahead up. For the active electrode placement as illustrated in illustration 618, there is a perimetry target of 620 which we propose to indicate right. For the active electrode placement as illustrated in illustration 622, there is a perimetry target of 624 which we propose to indicate left. For the active electrode placement as illustrated in illustration 626, there is a perimetry target of 628 which we propose to indicate look right. For the active electrode placement as illustrated in illustration 630, there is a perimetry target of 632 which we propose to indicate look left. For the active electrode placement as illustrated in illustration 634, there is a perimetry target of 636 which we propose to indicate backward down. For the active electrode placement as illustrated in illustration 638, there is a perimetry target of 640 which we propose to indicate do nothing.

### Study Design

Our study was designed in two phases: a pilot study and a feasibility study on human healthy volunteers.

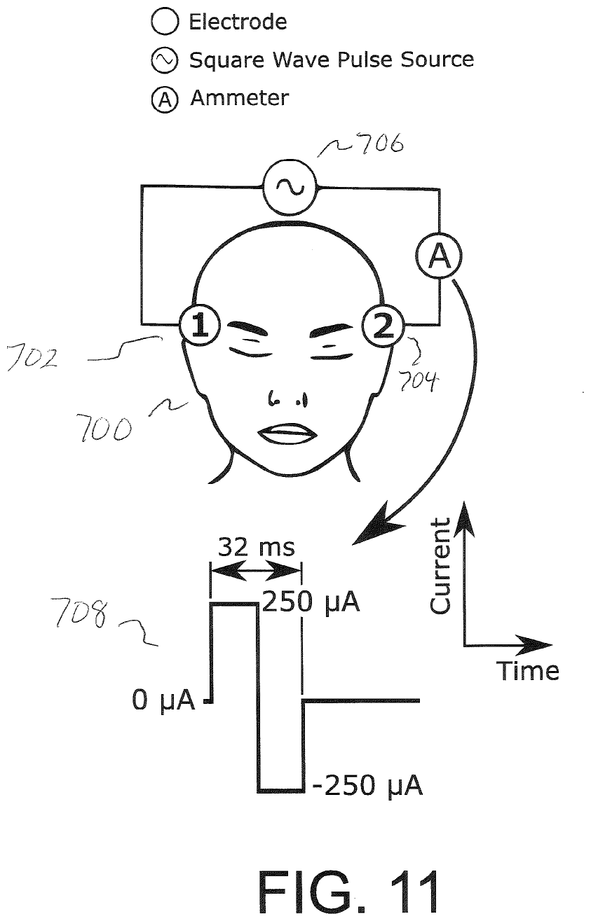

The purpose of the pilot study was to find an optimal set of electrical stimulation parameters namely the stimulation waveform shape, peak current intensity, and electrode size to reproducibly stimulate phosphenes at no skin irritation or pain. In this pilot study, a square pulse generator was wired across the temple of two healthy volunteers and an ammeter was placed in series with the associated circuit. Such a set up was optimized to observe the effectiveness of the stimulation to induce phosphenes reliably at absence of uncomfortable feeling. FIG. 11 (Pilot study) outlines the experimental setup of the pilot study.

Through a series of trial-and-error attempts, it was found that the electrical stimulation parameters outlined in FIG. 11 reliably produces phosphenes. After accomplishing the pilot stage, the next step was taken as described below.

Turning to FIG. 11, a person 700 has electrodes 702 and 704 attached to the temples as seen in FIG. 11. Person 700 keeps their eyes closed. A square wave pulse source 706 is activated resulting in the application of a charge-balanced square wave as defined in wave 708: a positive swing of +250 μA and a negative swing of −250 μA, and a waveform duration of 32 ms.

In a pilot study, the induced phosphenes demonstrated an associated spatial component according to subjective reporting from the subjects. The spatial component was reported to be in accordance with the location of the electrodes on the facial skin. For instance, the phosphenes that are stimulated by electrically stimulation of the skin regions om the right side of the face light up the right-most half of the visual field. Similarly, stimulating the left side of the face, generates phosphenes that light up the left side of the visual field.

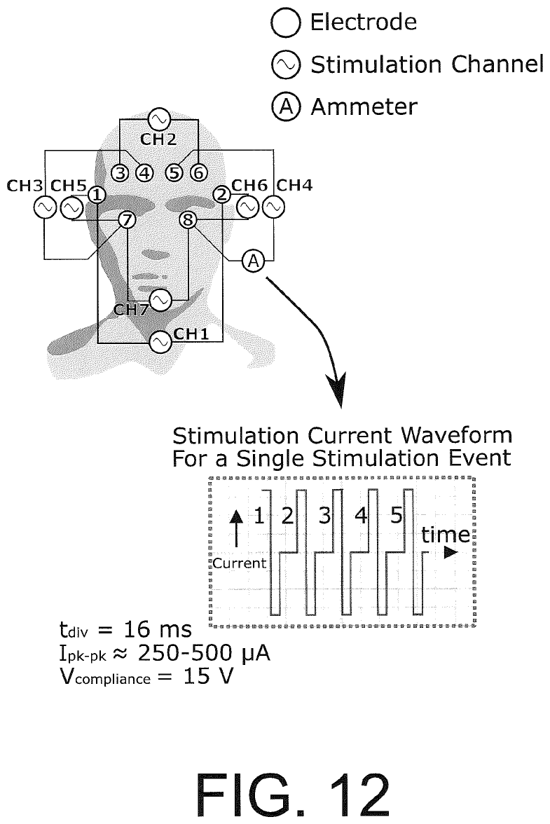

In order to have a data-driven approach to scientifically tackle such subjective observations, a human study was initiated where subjects would receive electrical stimulation across various pre-selected facial regions through numerous trial events, while they were to draw their percepts upon every electrical stimulation cycle. In order to run the human healthy volunteer study, a set of controlled and independent variables were defined. The control variables included an optimal set of electrical stimulation parameters namely the stimulation waveform shape, peak current intensity, and electrode size were determined. Through a series of trial-and-error attempts, it was found that the electrical stimulation parameters outlined in FIGS. 11 and 12 reliably produce phosphenes. Once found, such parameters were kept constant throughout the human study.

### Facial Electrodes' Placement

In the human study, each participant received the electrode dressing illustrated in FIG. 12. The dressing consisted of eight EEG gold cup electrodes that were arrayed around the orbital sockets, as shown in FIG. 4, using Top20™ conductive electrode paste and a 3 cm×3 cm gauze pad. The stimulation channels CH1, CH2, CH3, CH4, CH5, CH6, and CH7 were wired to the electrodes numbered 1 to 8 as shown in FIG. 12. A control channel was also devised, for use in control trials. During a control trial, no stimulation waveform would be present across any electrode. Turning to FIG. 12, eight skin electrodes 1, 2, 3, 4, 5, 6, 7, and 8 are positioned onto the head on the illustrated locations 1 through 8. Skin electrode 6 is positioned on the left side distally on the forehead and electrode 3 on the right side laterally on the forehead, and skin electrode 4 is positioned on the right side medially on the forehead and 5 is positioned on the left side medially onto the forehead. Skin electrode 7 is positioned on the right side of the person's head on the right cheekbone beneath the eyes to the right side of the nose bridge, and electrode 8 is positioned on the left side of the person's head on the left cheekbone beneath the eyes to the left side of the nose bridge. Skin electrodes 1 and 2 are positioned onto the right and left temples of the head respectively.

These skin electrodes are connected to stimulation channels CH1, CH2, CH3, CH4, CHS, CH6, Ch7 that are wired their respective skin electrodes. Specifically, channel CH1 has active electrodes 1 and 2 (the templar location); channel CH2 has active electrodes 3 and 6 (the distal forehead location); channel CH3 has active electrodes 4 and 7 (the right transorbital location); channel CH4 has active electrodes 5 and 8 (the left transorbital location); channel CH5 has active electrodes 1 and 7 (the right infraorbital location); channel CH6 has active electrodes 2 and 8 (the left infraorbital location); and channel CH7 has active electrodes 7 and 8 (the transnasal location).

The stimulation current waveform used is illustrated in FIG. 12, and is characterized as a tdiv of 16 ms, Ipeak to peak of approximately 250-500 μA, and a Voltage of 15 Volts.

### Experimental: Controllable Parameters

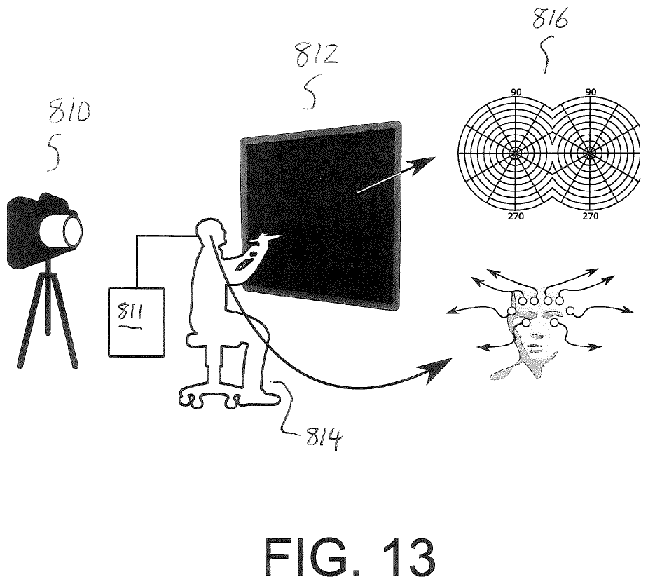

Study on human healthy volunteers was designed as a feasibility study conducted with 8 healthy individuals in order to map the phosphene location and shape across a healthy population as foundational research for future models. Eight participants were collected. Each participant signed a consent form and was then taken to a dimly lit room. Turning to FIG. 13, the participant 814 sits on a stool that was placed 50 cm away from a smartboard 812 displaying a ophthalmological binocular perimetry target 816. As illustrated in FIG. 13, a camera 810 was placed off-axis from the orthogonal axis of the smartboard 812. This allowed the participants' 810 drawings to be clearly photographed without the participant's body shadowing the scene. The participant would then receive the electrode dressing illustrated in FIG. 12 using EEG gold cup electrode. Before being placed onto the participant, each electrode was speared with Ten20™ electrode paste and then adhered to the participant's skin. A 3×3 cm gauze pad was placed on each electrode, thus enhancing the adhesive potency of the electrodes. The electrodes were connected to phosphene stimulator 811.

A preliminary step was performed, where the phosphene stimulation threshold was set for each participant before the remainder of the experiment. In this step, the templar electrode channel (CH1) stimulation was repeatedly applied whilst the stimulation current intensity was gradually raised. The participant was asked to report seeing a phosphene, which would signal the halting of raising of the stimulation current. The current level was always hardware restricted below 500 μA which is considered as safe for human involving research of head and brain. Once found, the stimulation intensity was kept constant for the remainder of the experiment. In our observations, the phosphene stimulations have not demonstrated fading effects requiring increasing the current.

After this preliminary stage, the data collection was initiated. During the data collection, stimulation sites, referred to as the stimulation channels (CH1, CH2, CH3, CH4, CH5, CH6, CH7 and CH8) were applied in a pseudorandomized fashion. To pseudo-randomize the stimulation channel sequencing, a MATLAB script was developed where numbers between 1 to 8 were pseudo-randomly organized and through a few conditional statements, it was made sure that each number was repeated for exactly 8 times, within the sequence. Such a sequence was hard coded into the stimulus administrator; therefore, the exact same sequence was used for each participant. Such a procedure made sure that the stimulation sequence would seem random to the participant, but its log was recorded.

Each channel was applied a total of 8 times, resulting in a total of 64 stimulation events. After each stimulation event, the participant 814 was asked to draw the contours of the phosphene they saw for that particular channel, from memory, on the smartboard 812 (FIG. 13). Each drawing was then accordingly photographed, using the camera.

### Experimental Setup: Variable Parameters

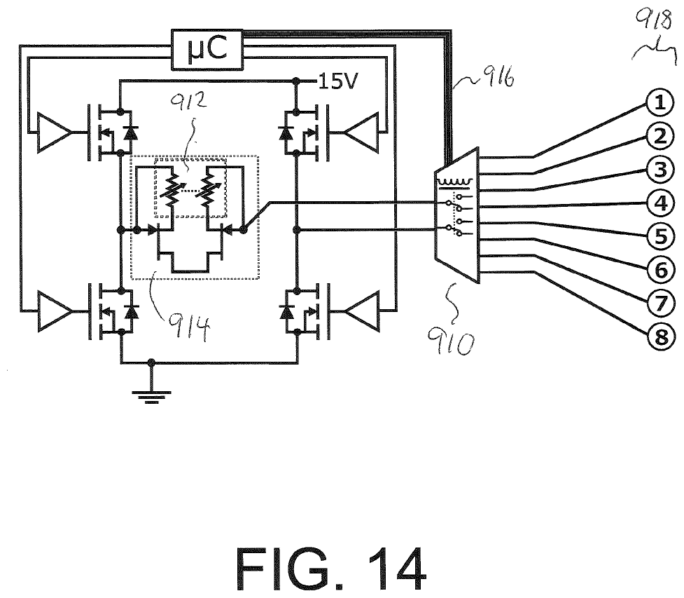

To limit the degrees of freedom, the only stimulation parameter that was varied was the electrode location. To administer the stimulation, a circuit was designed. A simple H-bridge circuit was constructed for the generation of the charge-neural waveform illustrated in FIG. 12. A DC voltage of 15V was used at the high-side of the H-bridge. This voltage was empirically found to be sufficient for supplying 250 to 500 μA across a wide range of skin impedances. An adjustable bipolar current limiting circuit was placed in series with one of the AC terminals of the H-bridge. The current limiter consisted of a common-drained pair of JFET adjustable current limiters, where a ganged potentiometer was used to couple and mirror the gate-source resistances of each JFET adjustable current limiter. This allowed for the continuous adjustment of the current limit, through the manual adjustment of an analog knob. The output of the current limiter and the other AC terminal of the H-bridge were then passed onto a double-pole relay network to demultiplex the stimulation current according to FIG. 12 across the 8 facial electrodes. A microcontroller was used to orchestrate the stimulation waveform generation and stimulation channel sequencing. The circuit architecture of the stimulus administrator is illustrated in FIG. 14. Turning to FIG. 14, the circuit includes an electromechanical relay demultiplexer, a coupled potentiometer 912, an adjustable current limiter 914, an 8-bit bus 916, and stimulation electrode 918.

### Data Collection

For each individual, each of the eight channels were energized eight times in a pseudo-randomized manner through 64 energization events lasting 200 milliseconds each whilst the smartboard displayed a binocular perimetry target. A perimetry target (FIGS. 9, 13) is mainly used in ophthalmology to spatially assess visual function. A binocular perimetry target displayed in FIG. 9 phenomenologically represents the joint visual field that is generated from both eyes. The used waveform (FIG. 12) consisted of five repetitions of the same charge-neutral waveform in order to ensure accurate noticeability of phosphenes for participants. After each stimulation event, participants 814 were asked to draw the remembered phosphene body contours on the associated smartboard 812 from memory. Phosphene maps were registered both as series of sketches from each individual and summarized as collective population phosphene maps. Results were recorded on camera for further data analysis. On average, each participant took 20 seconds to draw the recent percept from memory before the next electrode configuration is activated continuing until the 64th event's data will be collected.

The order of the channel energization events was pseudo-randomized, and each photograph was traced to its associated channel for the experiment conductors, even though to each participant the activation order of the electrodes was seemingly random and irregular in pattern for robust and unbiased data collection.

### Data Post-Processing

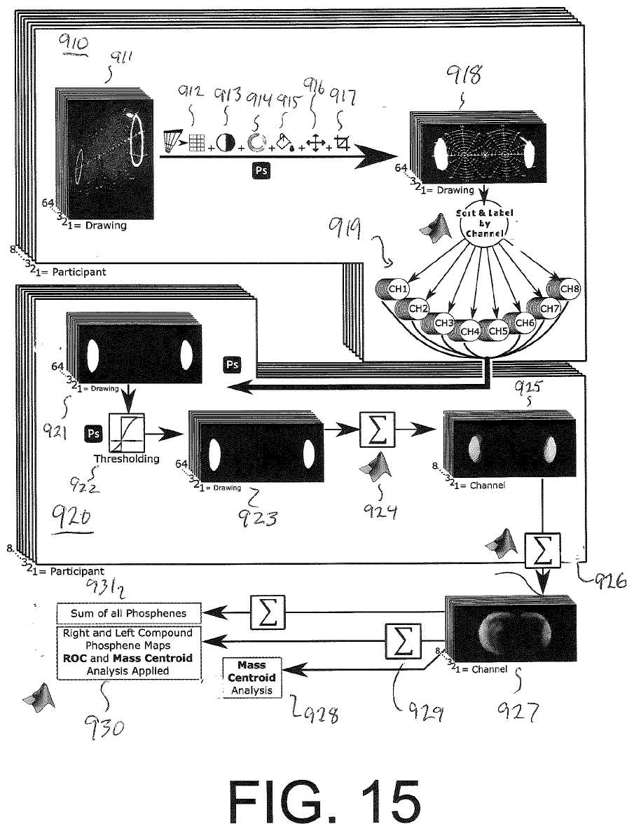

After the human trials, each participant produced 64 photographs with 512 photographs in total after the study for analysis. FIG. 15 displays the pre-processing and post-processing process flow to get the raw phosphene drawings to individual and population phosphene maps, and then to the Receive Operating Characteristic (ROC) and centroid analysis stage

The initial data was a photograph as per FIG. 15. Step 910 that have an orthographical perspective, with respect to the 64 photographs of drawings 911 performed by a participant. A series of manual and automated steps were applied to the each photograph—namely, perspective correction 912, contrast correction 913, color correction 914, image centering 916, and cropping 917 to correct for the perspective skew, glare, and positioning of the drawings. The contours in the drawings were shaded in converting the phosphene contours into phosphene body drawings 918. The pre-processed drawings were then labelled and categorized according to the stimulation channel that they're caused by (labelled 919).

In the following step 920, the phosphene drawings were isolated from their perimetry target background (step 921) and thereafter thresholded mid-scale (labelled 922) to get a binary mask 923 that represents the phosphene bodies that the participants perceived. So far, these steps were taken to pre-process the images from drawing photographs into illustrations that depict the phosphene percept bodies in the visual field. These steps were performed in Photoshop™ (Version 21.1.1, Adobe™, USA). Individual phosphene maps were generated by summing the phosphene drawings labelled under the same stimulation channel (labelled 924). A set of eight individual phosphene maps were generated for each participant. ROC analysis was applied to each individual phosphene map (labelled 925). Binary masks proposed in FIG. 10 in correspondence to each channel were used in the ROC analysis as the ground truth (GT).

A further step was taken by summing the individual phosphene maps belong to the same stimulation channel together (labelled 926), which resulted in the population phosphene maps 927. ROC analysis was applied to the population phosphene maps. Centroid analysis was applied to channels 2, and 7 (labelled 928). Centroid analysis was used to compare the difference of up (CH2) and down (CH7) population phosphene maps. As well, ROC analysis was applied to the population phosphene maps (labelled 929). Two compound phosphenes were generated by summing channels 3 and 5 (right) and channels 4 and 6 (left) channels. At this stage, right and left compound phosphene maps are generated and compared using ROC and mass centroid analysis (labelled 930). The up (CH2) and down (CH7) population phosphene maps were also compared using mass centroid analysis (labelled 928). The mass centroid analysis was done to compare phosphene maps that are used to implicitly encode up, down, right, and left directions to the participant.

A full summation of all of the phosphene drawings is computed, to see where the facial cutaneously stimulated phosphenes are spatially oriented (labelled 931). The entirety of steps involving individual, population, and compound phosphene map generation, as well as ROC and mass centroid analysis were done in MATLAB™ (Version R2020a, MathWorks™, USA).

### Statistical Analysis

For the analysis of the individual and collective phosphene maps, various metrics were needed to assess the proximity of the activated electrodes to the electrically-stimulated phosphene percept location. To achieve this, a ground truth mapping was developed, allowing the identification of the phosphene “visual” field (VF) regions in close proximity to their corresponding active electrodes through the consideration of the remainder background as false.

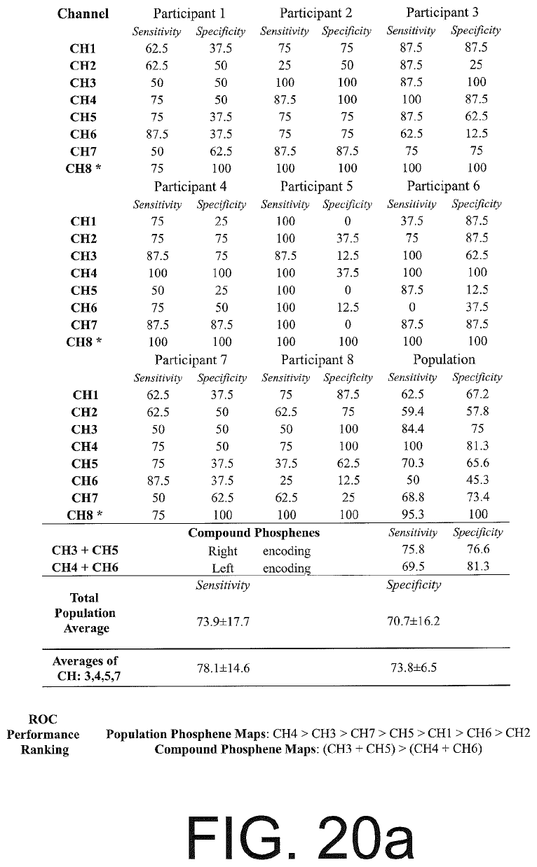

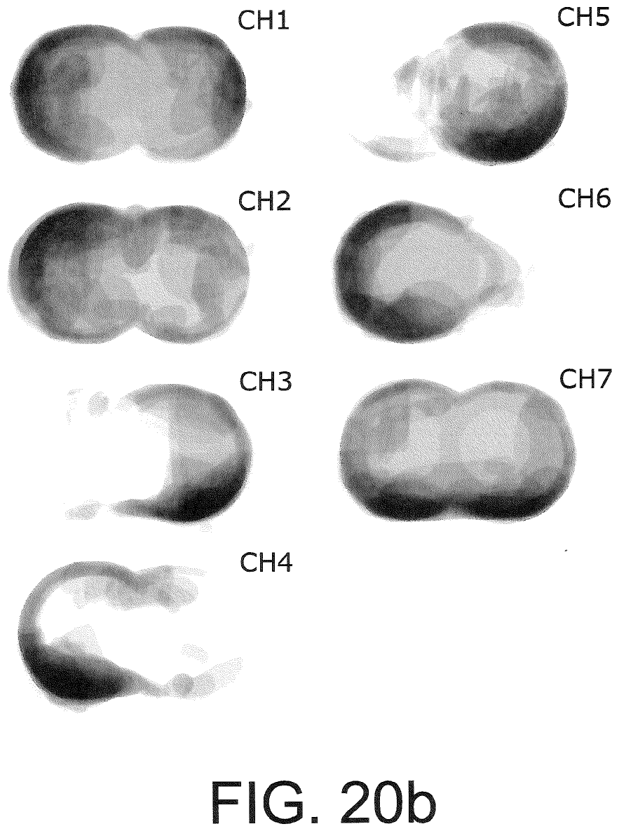

Sensitivity, a metric capable of measuring the ability of systems to detect a % of true positives, and specificity, a metric capable of measuring the ability of systems to detect a % of true negatives were used for the process. Both metrics (ROC and Mass Centroids) were computed through the two data sets of experimental results presented in the table (Human Study's Individual and Population Sensitivity and Specificity Values) in FIG. 20a and ground truth to create the four sub-variable figures of true positive, false positive, true negative, and false negative. A maximum score of 8 per channel was achievable for the individual phosphene maps while a maximum score of 64 per channel was possible for the population phosphene maps.

For the finalization of results, the phosphene marked regions 1010 in FIG. 10 were assigned a value of one whilst the background regions 1012 in FIG. 2 were assigned a value of zero in order to create the ground truth maps. (Ground truths outline where we expect to see phosphenes for a particular electrode config. Ground truth maps illustrate the regions of expectation and lack of expectation as a binary 2D array where regions marked with a value of 1 indicate regions of expectation and markings of 0 indicate the absence of expectation of phosphene perception.)

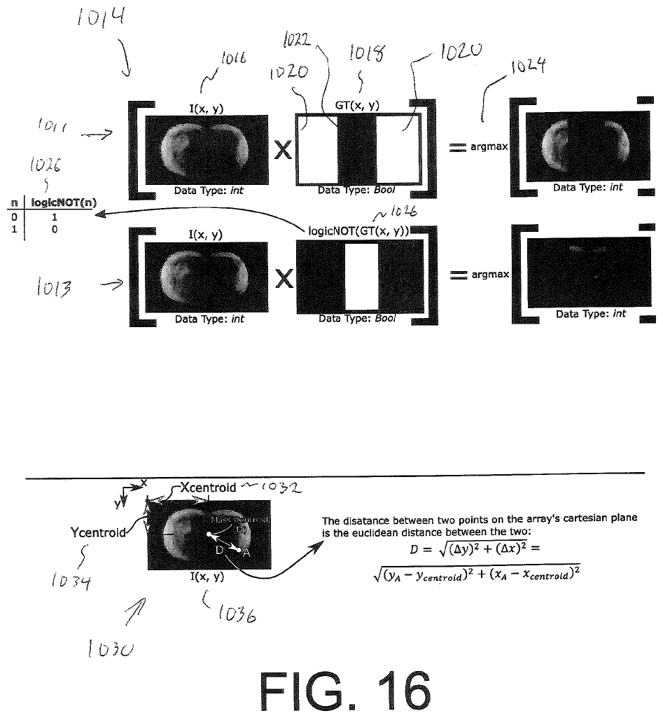

FIG. 16 illustrates an example of the ROC analysis being computed on one of the population phosphene maps. Turning to FIG. 16, there is an ROC analysis example 1014. Equations 1 to 6 were used to compute ROC metrics of sensitivity and specificity. Example 1011 is a True Positive=argmax, and Example 1013 is a False Positive=argmax. The term “I(x, y)” 1016 refers to the channel specific phosphene map. Such a phosphene map could be individual, population, or compound phosphene map. The term “GT(x, y)” 1018 is the channel specific ground truth map that is derived from the hypothesized expectation of the results. The ground truth is a binary mask that marks the areas of phosphene expectation with a logic 1 (high) 1020 and the areas where phosphenes are unexpected with a logic 0 (low) 1022. The “argmax” 1024 is a statistical operation where the resultant is the magnitude of the largest array element within its argument. (the expected value for argmax is an integer between 0 to 8 for individual phosphene maps; an integer between 0 to 64 for population phosphene maps; and an integer between 0 to 128 for compound phosphene maps) The “logicNOT” 1026 is a Boolean operation where the logic states of each element in the array is flipped. This entails flipping all logic highs to low and all logic lows to high within the array of its argument. The “MaxScore” is the maximum grey value possible within each class of phosphene map. (the MaxScore is constant for each class of phosphene maps; for individual phosphene maps the MaxScore is 8; for population phosphene maps the MaxScore is 64; and for compound phosphene maps the MaxScore is 128) The maximum possible score for individual, population, and compound phosphene maps are 8, 64, and 128 respectively. False negative=MaxScore−true positive, while true negative=MaxScore−false positive.

$$True Positive = arg max ( I ( x , y ) \times GT ( x , y ) ) \qquad Eq . 1$$

$$False Positive = arg max ( I ( x , y ) \times logicNOT ( GT ( x , y ) ) ) \qquad Eq . 2$$

$$False Negative = MaxScore - True Positive \qquad Eq . 3$$

$$True Negative = MaxScore - False Positive \qquad Eq . 4$$

$$Sensitivity = ( \frac{True Positive}{True Positive + False Negative} ) \qquad Eq . 5$$

$$Specificity = ( \frac{True Negative}{True Negative + False Positive} ) \qquad Eq . 6$$

Eq. 7 was used to assess the efficacy of each channel in inducing phosphenes. This equation can also be used to assess the efficacy of the control channel. A low percentage on the efficacy of the control channel would suggest the control the false positive immunity of our methodology to induce phosphenes. In Eq.7 the “I” term represents the individual phosphene maps particular to a stimulation channel. The “Max_Score” term is 8, since that's the maximum score possible for each individual phosphene map. Lastly, the “N” term is the population size, which is 8. The “i” term is the iterator that looks through the members of the population.

$$Likelihood = \frac{{\sum }_{i = 1}^{N} ( \frac{max ( I ( x , y , i ) )}{Max\_Score} )}{N} \times 100 \% \qquad Eq . 7$$

The secondary analysis that was used to assess the discriminability of phosphene maps is mass centroid analysis 1030. Specifically, such an analysis was used to assess the discriminability of left and right encoding phosphenes, as well as the up and down encoding phosphenes. Eq. 8 and Eq. 9 were used to compute the vertical 1032 and horizontal 1034 coordinates of the spatial mass centroid of population phosphene maps during centroid analysis. The term “I(x, y)” 1036 is the phosphene map 2D array. The terms “x” and “y” represent the x and y coordinates within the 2D cartesian array.

The computation method of the mass centroid of the phosphene map pixels is very similar to that of in physics. Each pixel is assumed to have a point mass to that of its grey value and a two-dimensional cartesian coordinate equal to that of its x and y indices in the array. The x-centroid of the pixel array can be found by first computing the 2D summation of the product of each pixel's grey value and its corresponding x-coordinate. Diving the resultant by the summation of all grey values within the array, will yield the x-centroid. The y-centroid can be found in a similar manner, with the difference in the initial term, where the grey values are multiplied by their corresponding y-coordinate instead.

$${X}_{Centroid} = ( {\sum }_{all x} {\sum }_{all y} ( I ( x , y ) \times x ) ) \div ( {\sum }_{all x} {\sum }_{all y} I ( x , y ) ) \qquad Eq . 8$$

$${Y}_{Centroid} = ( {\sum }_{all x} {\sum }_{all y} ( I ( x , y ) \times y ) ) \div ( {\sum }_{all x} {\sum }_{all y} I ( x , y ) ) \qquad Eq . 9$$

In order to compare the outcomes of the mass centroid analysis, Euclidean distance is used to assess the discriminability between the phosphenes. It is assumed that phosphenes that possess farther centroids are more spatially discriminable. Eq. 10 illustrates the equation expressing Euclidean distance. The “Δy” term is the difference of the y-coordinates of the two centroids and the “Δx” term is the difference of the x-coordinates of the two centroids. The term “D” represents the magnitude of the Euclidean distance. FIG. 16 illustrates how the mass centroid analysis is performed and how the mass centroids are compared.

D=\sqrt{(Δy)²+(Δx)²} Eq.10

When ranking the performance of each population phosphene map, the numerical figure that was used to sort the rankings was assumed to be the sum of sensitivity and specificity figures for each channel. Eq. 11 illustrates the approach to such a metric.

Ranking Score=Sensitivity+Specificity Eq. 11

Lastly, Due to the similarities of the hypothesized ground truths of channels CH3 and CH5, as well as CH4 and CH6, a set of compound phosphenes maps were generated, where a right encoding compound phosphene map was generated by following Eq. 12 and a left encoding compound phosphene map was generated by following Eq. 13. The phosphene maps used to generate such phosphene maps were the population phosphene maps. ROC and centroid analyses was used on both of such compound phosphene maps, to compare the performances of the two against themselves, as well as against the up (CH2) and down (CH7) population phosphene maps.

Right Compound Phosphene Map=I(x, y)_{CH3}+I(x, y)_{CH5} Eq. 12

Left Compound Phosphene Map=I(x, y)_{CH4}+I(x, y)_{CH6} Eq. 13

In the compound phosphene map ROC analyses, the ground truth (GT) map of CH3 was used to assess the tight encoding compound phosphene map and the CH4 ground truth map from FIG. 2 was used to obtain the sensitivity and specificity figures. This is justifiable since the ground truth map of CH3 expects phosphene in the right-half of the visual field, while the GT for CH4 expects phosphenes in the left-half of the visual field.

Lastly, to compute the total sum of phosphenes to depict the overall manifestations of the phosphenes generated by our proposed apparatus, Eq. 14 is used to compute such a sum.

Total sum=Σ_{n=1,2,3, . . . , 8} I(x, y)_{CH}_{ₙ} Eq.14

### Results and Discussion

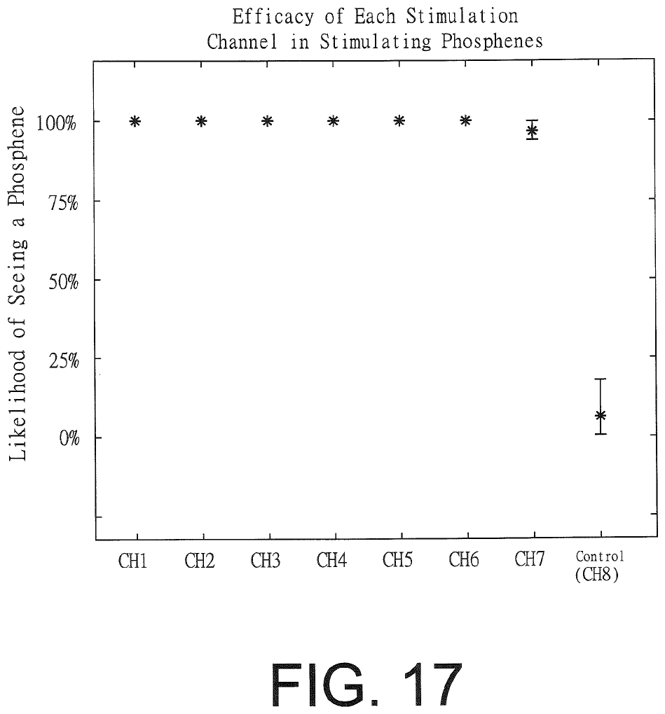

The waveforms shown in FIGS. 11 and 12 generated by the channels CH1 through CH7 were all statistically significant in phosphene induction in the human study. Each channel was assessed to effectively and reproducibly produce localized phosphenes. The efficacy of producing phosphenes (regardless of spatial coding) that is the ratio of phosphene stimulation success versus the total number of stimulation attempts, %) was 99±1% for every single channel among CH1 through CH7. FIG. 17 displays the efficacy of each stimulation channel, as well as the control (CH8) in stimulating phosphenes.

The ability of each phosphene map to stay true to its hypothesized outcome according to FIGS. 9 and 10 and measured through the metrics of sensitivity and specificity was found varied across the tested population. For the channels CH1 through CH7 and HC8 shown in FIGS. 10 and 11, the ROC analysis metrics of average sum of the sensitivity and specificity score has been found to be 156 (out of max 200) for the entire population of the study. From those, we found 2 distinct groups of 4 persons each with the scores 114 and 198 consequently, which may reflect a natural ability for the phosphene's perception. Conditionally we named these 2 groups as “strong phosphene responsive group” and “weak phosphene responsive group” or just as the strong and weak groups.

Overall (turning to the table in FIG. 20a), the proposed electrode configuration for the channels CH1 through CH8 produced average sensitivity and specificity scores of 73.9% and 70.7% respectively. The control trial was 80% effective in producing no phosphenes. While we do not know the reason why the existing stimulation configuration and electrode placements produced spatially resolved true positives (according to FIG. 17) less than 100%, we can hypothesize that it can be dependent on natural ability to percept phosphenes. It is confirmed by 79.7%/72.7% sensitivity/specificity for the strong group versus 68.1%/68.7% for the weak group, although both the strong and weak groups demonstrated statistically significant results.

The population phosphene maps are illustrated in FIG. 20b for CH1 through CH7. In FIG. 20b, the observed phosphenes are indicated by the darker sections of the

The ability of each phosphene map to stay true to its hypothesized outcome according to FIGS. 9 and 10 and measured through the metrics of sensitivity and specificity was found varied across the tested population. For the channels CH1 through CH7 and CH8 shown in FIGS. 10 and 11, the ROC analysis metrics of average sum of the sensitivity and specificity score has been found to be 156 (out of max 200) for entire population of the study. From those, we found 2 distinct groups of 4 persons each with the scores 114 and 198 consequently, which may reflect a natural ability for the phosphene's perception. Conditionally we named these 2 groups as “strong phosphene responsive group” and “weak phosphene responsive group” or just as the strong and weak groups.

Overall (turning to the table in FIG. 20a), the proposed electrode configuration for the channels CH1 through CH7 and CH8 produced average sensitivity and specificity scores of 73.9% and 70.7% respectively. The control trial was 80% effective in producing no phosphenes. While we do not know the reason why the existing stimulation configuration and electrode placements produced spatially resolved true positives (according to FIGS. 20a) less than 100%, we can hypothesize that it can be dependent on natural ability to percept phosphenes. It is confirmed by 79.7%/72.7% sensitivity/specificity for the strong group versus 68.1%/68.7% for the weak group, although both the strong and weak groups demonstrated statistically significant results.

In terms of channel-based ROC analysis, channels 3 and 4 had the highest ROC score, while channels 8 and 6 came in third and fourth place respectively. Channels 3 and 4 displayed the highest sensitivity/specificity (84.4%/75% (CH3) and 100%/81.3% (CH4)), Channels 6 and 7 displayed less sensitivity/specificity, while channels 1, 5, and then 2 followed the remainder of the ROC performance ranking from higher to lower. The table in FIG. 20a outlines the sensitivity and specificity values of all individual, population, and compound phosphenes. CH2 population phosphenes were found to contain the lowest quality ROC performance in terms of sensitivity and specificity. It was found through further testing that despite the initial experiment hypothesis, the electrically-stimulated phosphenes do not always appear in close proximity to the stimulating electrodes. For the best performing phosphene maps (CH3, 4, 5, 8), ROC analysis suggested that phosphenes do appear near the stimulation electrodes, with an average sensitivity and specificity score of 73.9% and 70.7% respectively. Adhering to the ground truth maps, the left and right oriented compound phosphene map pairs possessed a higher ROC score than the up and down oriented phosphene map pairs.

Such combinations were implemented through summing the CH3/CH5 (right) and CH4/CH6 (left) phosphene maps which gives 75.8%/76.6% sensitivity/specificity for the right encoded compound channels, versus the 69.5%/81.3% sensitivity/specificity of the left encoded compound phosphene map.

This can be interpreted as the up/down phosphene map pairs sensitivity and specificity is lower than the right/left phosphene map pair and we can speculate that up/down phosphene map pairs are less discriminable comparing the left/right phosphene coding at the used location of the electrodes.

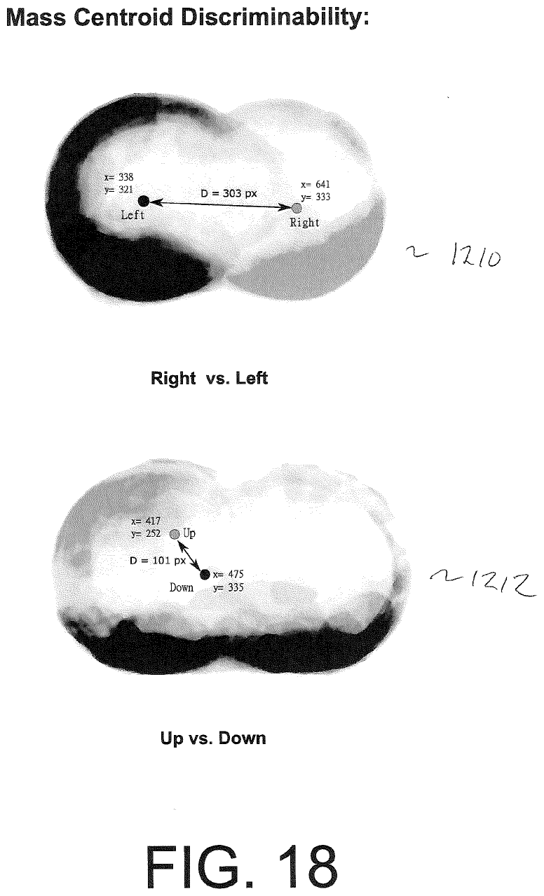

The mass centroid analysis suggests the same outcome. Turning to FIG. 18, left and right compound phosphene maps have more spaced apart centroids than the up and down population phosphene maps. Assuming that the centroid distance between two phosphene maps is representative of the discriminability of the two phosphenes, then left and right compound phosphenes are more discriminable. As depicted in FIG. 18, the left and right compound phosphenes possess an intra-centroid distance of 303 pixels, while the up and down population phosphene maps have an intra-centroid distance of only 101 pixels. Turning to FIG. 18, the mass centroid discriminability for right vs left oriented phosphenes: CH3+CH5 vs. CH$+CH6 is shown in 1210, and the centroid discriminability for up vs down oriented phosphenes: CH2 vs. CH7, is shown in 1212. Such an intra-centroid distance disparity agrees with the conclusions made from the ROC analysis, where the left and right compound phosphene maps perform better according to the metrics of sensitivity and specificity in comparison to up and down (CH2 and CH7 respectively) population phosphene maps.

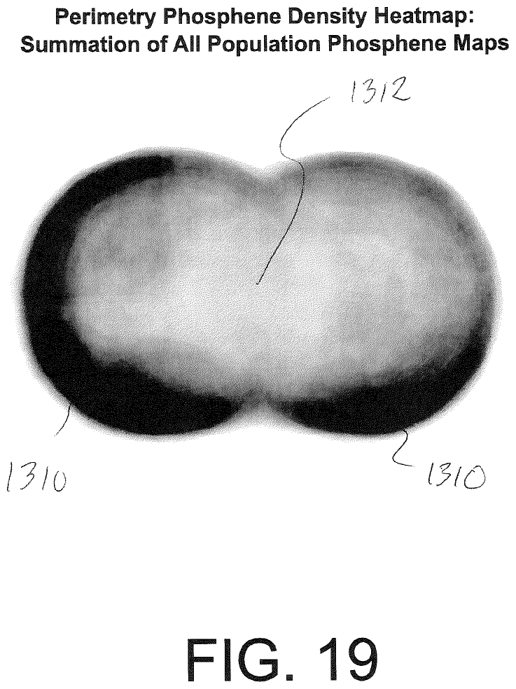

After summing the entirety of the population phosphene maps into a cumulative phosphene map, it was found that phosphenes stimulated through our method have a tendency to appear in the peripheral visual field similar to what can be FIG. 19. The central visual field shows the general absence of phosphenes according to the collected phosphene drawings. Turning to FIG. 19, there are areas of high phosphene density 1310 and areas of low phosphene density 1312. It is important to note than accessing the central visual field has the advantage of increasing the information bandwidth between the participant and the stimulus administrator.

FIGS. 1 through 20b are conceptual illustrations allowing for an explanation of the present invention. Those of skill in the art should understand that various aspects of the embodiments of the present invention could be implemented using different materials and minor design modifications. Notably, the figures and examples above are not meant to limit the scope of the present invention to a single embodiment, as other embodiments are possible by way of interchange of some of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention.

1. A system comprising:
One to eight pairs of electrodes for cutaneous connection to a person, where the one to eight pairs of electrodes when connected to the person are located at a location selected from the group consisting of:
the right temple of the person's head and the left temple of the person's head; or
the left temple of the person's head and the left side of the person's head bilaterally across the nose bridge beneath the eyes; or
the right temple of the person's head and the right side of the person's head bilaterally across the nose bridge beneath the eyes; or
the right side of the person's head on the right cheekbone beneath the eyes to the right side of the nose bridge and the left side of the person's head on the left cheekbone beneath the eyes to the left side of the nose bridge; or
the right side medially on the person's head and the left side medially on the person's head; or
the left side medially on the person's head and the left side of the person's head on the left cheekbone beneath the eyes to the left side of the nose bridge; or
the right side medially on the person's head and the right side of the person's head on the right cheekbone beneath the eyes to the right side of the nose bridge; or
the right side laterally on the person's head and the left side distally on the person's head;
Each of the one to eight pairs of electrodes being connected to a waveform generator;
The waveform generators being connected to a first control device configured to activate a voltage waveform between the one to eight pairs of electrodes;
The system being connected to a power source.

2-19. (canceled)

20. A system comprising:
One to sixteen electrodes, where the one to sixteen electrodes when connected to a person are located at a location selected from the group consisting of:
the left temple of the head; or
the right temple of the head; or
under the left eye at an interior position; or
under the left eye at a middle position; or
under the left eye at an exterior position; or
under the right eye at an interior position; or
under the right eye at a middle position; or
under the right eye at an exterior position; or
above the left eye at an interior position; or
above the left eye at a middle position; or
above the left eye at an exterior position; or
above the right eye at an interior position; or
above the right eye at a middle position; or
above the right eye at an exterior position; or
above the nose to the right side; or
above the nose to the left side;
Where each of the one to sixteen electrodes form a unipolar pair with a seventeenth electrode, where the seventeenth electrode can be cutaneously connected to the person;
Each of the one to sixteen electrode pairs being connected to a waveform generator;
The waveform generators being connected to a first control device configured to activate a voltage waveform between the each of the one to sixteen electrodes and the seventeenth electrode; and
The system being connected to a power source.

21-31. (canceled)

32. A method of generating phosphenes in a person, comprising:
Cutaneously attaching one to eight pairs of electrodes to a person, where the one to eight pairs of electrodes whose locations are selected from the group consisting of:
the right temple of the person's head and the left temple of the person's head; or
the left temple of the person's head and the left side of the person's head bilaterally across the nose bridge beneath the eyes; or
the right temple of the person's head and the right side of the person's head bilaterally across the nose bridge beneath the eyes; or
the right side of the person's head on the right cheekbone beneath the eyes to the right side of the nose bridge and the left side of the person's head on the left cheekbone beneath the eyes to the left side of the nose bridge; or
the right side medially on the person's head and the left side medially on the person's head; or
the left side medially on the person's head and the left side of the person's head on the left cheekbone beneath the eyes to the left side of the nose bridge; or
the right side medially on the person's head and the right side of the person's head on the right cheekbone beneath the eyes to the right side of the nose bridge; or
the right side laterally on the person's head and the left side distally on the person's head;
Where each of the one to eight pairs of electrodes is connected to a waveform generator; and the waveform generators are connected to a first control device configured to activate at least one voltage waveform between the one to eight pairs of electrodes;
Sending input data to the first control device, the first control device converting the input data to a pattern of activation for the one to eight pairs of electrodes, and so activating the one to eight pairs of electrodes to generate a phosphene pattern in the person using the following pattern:
activating the electrode pair at the right temple of the person's head and the left temple of the person's head to create a phosphene in in left and right sides of the visual field of the person;
activating the electrode pair at the left temple of the person's head and the left side of the person's head bilaterally across the nose bridge beneath the eyes to create a phosphene in the lower left of the visual field of the person;
activating the electrode pair at the right temple of the person's head and the right side of the person's head bilaterally across the nose bridge beneath the eyes to create a phosphene in the lower right of the visual field of the person;
activating the electrode pair at the right side of the person's head on the right cheekbone beneath the eyes to the right side of the nose bridge and the left side of the person's head on the left cheekbone beneath the eyes to the left side of the nose bridge to create a phosphene in the bottom of the visual field of the person;
activating the electrode pair at the right side medially on the person's head and the left side medially on the person's head to create a phosphene in the top of the visual field of the person;
activating the electrode pair at the left side medially on the person's head and the left side of the person's head on the left cheekbone beneath the eyes to the left side of the nose bridge to create a phosphene in the middle left of the visual field of the person;
activating the electrode pair at the right side medially on the person's head and the right side of the person's head on the right cheekbone beneath the eyes to the right side of the nose bridge to create a phosphene in the middle right of the visual field of the person;
activating the electrode pair at the right side laterally on the person's head and the left side distally on the person's head to create a phosphene in the upper right and upper left of the visual field of the person.

33-58. (canceled)

59. The system of claim 1 where the first control device is configured to receive input data and convert the input data to a pattern of activation for the one to eight pairs of electrodes using the following pattern:
activating the electrode pair at the right temple of the person's head and the left temple of the person's head to create a phosphene in in left and right sides of the visual field of the person;
activating the electrode pair at the left temple of the person's head and the left side of the person's head bilaterally across the nose bridge beneath the eyes to create a phosphene in the lower left of the visual field of the person;
activating the electrode pair at the right temple of the person's head and the right side of the person's head bilaterally across the nose bridge beneath the eyes to create a phosphene in the lower right of the visual field of the person;
activating the electrode pair at the right side of the person's head on the right cheekbone beneath the eyes to the right side of the nose bridge and the left side of the person's head on the left cheekbone beneath the eyes to the left side of the nose bridge to create a phosphene in the bottom of the visual field of the person;
activating the electrode pair at the right side medially on the person's head and the left side medially on the person's head to create a phosphene in the top of the visual field of the person;
activating the electrode pair at the left side medially on the person's head and the left side of the person's head on the left cheekbone beneath the eyes to the left side of the nose bridge to create a phosphene in the middle left of the visual field of the person;
activating the electrode pair at the right side medially on the person's head and the right side of the person's head on the right cheekbone beneath the eyes to the right side of the nose bridge to create a phosphene in the middle right of the visual field of the person;
activating the electrode pair at the right side laterally on the person's head and the left side distally on the person's head to create a phosphene in the upper right and upper left of the visual field of the person.

60. The system of claim 59 where the pattern further comprises activating two pairs of electrodes simultaneously to create centroids.

61. The system of claim 60 where the activated two pairs of electrodes have identical electrical stimulation intensities.

62. The system of claim 60 where the activated two pairs of electrodes have different electrical stimulation intensities.

63. The system of claim 60 further comprising a sensor, the sensor being configured to send input data to the first control device, where the sensor is a spatial sensor and the input data is spatial data and the first control device converts the spatial data into a phosphene pattern that, when the two to eight electrodes are connected to the person, communicates information about the person's spatial surroundings to the person.

64. The system of claim 59 where the voltage waveform is a biphasic square signal.

65. The system of claim 20 where the first control device is configured to receive input data and convert the input data to a pattern of activation for the one to sixteen electrodes using the following pattern:
activating the electrode at the left temple of the head to create a phosphene in the middle left of the visual field of the person;
activating the electrode at the right temple of the head to create a phosphene in the middle right of the visual field of the person;
activating the electrode under the left eye at an interior position to create a phosphene in the lower right of the visual field of the person;
activating the electrode under the left eye at a middle position to create a phosphene in the middle left of the visual field of the person;
activating the electrode under the left eye at an exterior position to create a phosphene in the bottom left of the visual field of the person;
activating the electrode under the right eye at an interior position to create a phosphene in the middle top of the visual field of the person;
activating the electrode under the right eye at a middle position to create a phosphene in the middle right of the visual field of the person;
activating the electrode under the right eye at an exterior position to create a phosphene in the bottom right of the visual field of the person;
activating the electrode above the left eye at an interior position to create a phosphene in the left middle of the visual field of the person;
activating the electrode above the left eye at a middle position to create a phosphene in the top left of the visual field of the person;
activating the electrode above the left eye at an exterior position to create a phosphene in the left side of the visual field of the person;
activating the electrode above the right eye at an interior position to create a phosphene in the middle right of the visual field of the person;
activating the electrode above the right eye at a middle position to create a phosphene in the top right of the visual field of the person;
activating the electrode above the right eye at an exterior position to create a phosphene in the right side of the visual field of the person;
activating the electrode above the nose to the right side to create a phosphene in the top left to middle of the visual field of the person;
activating the electrode above the nose to the left side to create a phosphene in the top right to middle of the visual field of the person.

66. The system of claim 65 where the pattern further comprises activating two electrodes simultaneously to create centroids.

67. The system of claim 66 where the activated two electrodes have identical electrical stimulation intensities.

68. The system of claim 66 where the activated two electrodes have different electrical stimulation intensities.

69. The system of claim 66 further comprising a sensor, the sensor being configured to send input data to the first control device, where the sensor is a spatial sensor and the input data is spatial data and the first control device converts the spatial data into a phosphene pattern that, when the two to eight electrodes are connected to the person, communicates information about the person's spatial surroundings to the person.

70. The system of claim 65 where the voltage waveform is a biphasic square signal.

71. The method of claim 32 where the pattern further comprises activating two pairs of electrodes simultaneously to create centroids.

72. The method of claim 71 where the activated two pairs of electrodes have identical electrical stimulation intensities.

73. The method of claim 71 where the activated two pairs of electrodes have different electrical stimulation intensities.

74. The method of claim 71 where the input data further comprises spatial data from a spatial sensor and the first control device converts the spatial data into a phosphene pattern that, when the two to eight electrodes are connected to the person, communicates information about the person's spatial surroundings to the person.
